SEQUENCE LISTING (16 sequences)

SEQ ID NO: 1 - protein, 115 aa, Ixodes scapularis
MTGVFGGYSERANHQANPEFLNLAHYATSTWSAQQPGKTHFDTVAEVVKVETQVVAGTNYRLTLKVAESTCELTSTYNKDTCLPKADAAHRTCTTVVFENLQGDKSVSPFECEAA

SEQ ID NO: 2 - protein, 115 aa, Ixodes scapularis
MELALRGGYRERSNQDDPEYLELAHYATSTWSAQQPGKTHFDTVVEVLKVETQTVAGTNYRLTLKVAESTCELTSTYNKDTCQANANAAQRTCTTVIYRNLQGEKSISSFECAAA

SEQ ID NO: 3 - protein, 19 aa, Ixodes scapularis
MTSTFALVLLLGGMAVCVA

SEQ ID NO: 4 - protein, 18 aa, Ixodes scapularis
MTSSLALVLLFGGAAVCA

SEQ ID NO: 5 - DNA, 41 bp, Artificial Sequence
  Description of Artificial Sequence Synthetic primer
gcccatatggaactggcactgcgtggcggttaccgcgagcg

SEQ ID NO: 6 - DNA, 36 bp, Artificial Sequence
  Description of Artificial Sequence Synthetic primer
gccctcgagttatgcggccgcacactcaaaggagct

SEQ ID NO: 7 - DNA, 24 bp, Artificial Sequence
  Description of Artificial Sequence Synthetic primer
ctatgcggcttcctcgaaggggct

SEQ ID NO: 8 - DNA, 27 bp, Artificial Sequence
  Description of Artificial Sequence Synthetic primer
ggctacagcgagagggcgaaccaccaa

SEQ ID NO: 9 - DNA, 24 bp, Artificial Sequence
  Description of Artificial Sequence Synthetic primer
agcgaagacggtctcgagcaagat

SEQ ID NO: 10 - DNA, 24 bp, Artificial Sequence
  Description of Artificial Sequence Synthetic primer
tcggcacacgatgcctcagggaat

SEQ ID NO: 11 - DNA, 24 bp, Artificial Sequence
  Description of Artificial Sequence Synthetic primer
gaagatcttgagaagatggcccag

SEQ ID NO: 12 - DNA, 20 bp, Artificial Sequence
  Description of Artificial Sequence Synthetic primer
cggtaccgtcgatggtcacc

SEQ ID NO: 13 - DNA, 23 bp, Artificial Sequence
  Description of Artificial Sequence Synthetic primer
tcgcgatcgctagcatcacactt

SEQ ID NO: 14 - DNA, 24 bp, Artificial Sequence
  Description of Artificial Sequence Synthetic primer
agcagaaggaccaaagcgaaggta

SEQ ID NO: 15 - DNA, 25 bp, Artificial Sequence
  Description of Artificial Sequence Synthetic primer
aagtccattagctccttcgagtgtg

SEQ ID NO: 16 - DNA, 24 bp, Artificial Sequence
  Description of Artificial Sequence Synthetic primer
atcattccgcgacgtacagtgaga

(19) United States
(12) Patent Application Publication
(10) Pub. No.: US 2010/0278752 A1
(43) Pub. Date: Nov. 4, 2010

(54) Methods for Detection and Prevention of Tick Infestation and Pathogen Transmission

(75) Inventors: Michail Kotsyfakis, Ceske Budejovice (CZ); Jose M.C. Ribeiro, Rockville, MD (US); Jesus G. Valenzuela, Gaithersburg, MD (US); John F. Andersen, Kensington, MD (US); Jennifer M. Anderson, Washington, DC (US); Shahid Karim, Hattiesburg, MS (US); Thomas N. Mather, Kingston, RI (US)

Correspondence Address: University of Rhode Island;Division of Reasearch and Economic Development, 75 Lower College Road, Suite 001, Kingston, RI, 02881, US

(73) Assignee: Rhode Island Board of Governors for Higher Education, Kingston, RI (US)

(21) Appl. No.: 12/671,703

(22) PCT Filed: Jul. 25, 2008
(86) PCT No.: PCT/US08/09075

§ 371 Date: Jul. 8, 2010

Related U.S. Application Data
(60) Provisional application No. 60/963,332, filed on Aug. 2, 2007.

(51) Int. Cl. A61B 5/00; A61K 39/00

(52) U.S. Cl. 424/9.81; 424/185.1

(57) ABSTRACT

The invention generally features methods for the prevention and detection of a tick infestation. The present invention also features methods for decreasing the ability of a tick to feed on a subject.

### RELATED APPLICATIONS

This application claims priority from U.S. Provisional Application No. 60/963,332 filed Aug. 2, 2007, and which is incorporated herein by reference in its entirety.

### STATEMENT OF RIGHTS TO INVENTIONS MADE UNDER FEDERALLY SPONSORED RESEARCH

This work was supported by the following grants from the National Institutes of Health, Intramural Grant No: 8334319, Extramural Grant No: 5R01AI037230. The government may have certain rights in the invention.

### BACKGROUND OF THE INVENTION

The incidence of tick-borne diseases has drastically increased over the past few years and according to the Center for Diseases Control (CDC), Lyme disease is one of the fastest-growing infectious diseases in the United States (US) reaching 23,000 reported cases for 2005.

Lyme disease is transmitted by the bite of Ixodes ticks. While taking a blood meal, ticks are attached to their host for several days and introduce saliva into the host skin that contains a wide range of physiologically active molecules, crucial for attachment to the host or for the transmission of pathogens. Infection is caused by the bacterium Borrelia burgdorferi resulting in a chronic, progressive infection which attacks many organs, such as the skin, the central and peripheral nervous system, the heart, the liver, the kidneys and musculoskeletal system. A key to avoiding serious effects of infection is prompt diagnosis and treatment of the underlying disorder. However, early detection of Lyme disease is difficult because the characteristic rash is not evident, and the flu-like symptoms which can be caused by many other factors complicate proper diagnosis. Moreover, many current assays used in laboratories are unreliable.

Accordingly, compositions and methods for detecting and treating Lyme disease are needed.

### SUMMARY OF THE INVENTION

As described below, the present invention features compositions and methods for preventing and detecting tick infestation in a subject. Additionally, the invention features controlling blood feeding in the tick.

In one aspect, the invention provides a method for the detection of a tick infestation in a subject comprising administering to the subject an immunogenic composition comprising one or more cystatin antigens, and thereby detecting a tick infestation in a subject.

In certain embodiments, the method further comprises the step of monitoring the subject for a response. In further embodiments, the response is an inflammatory response.

In another aspect, the invention provides a method for the detection of a tick infestation in a subject comprising administering to the subject an immunogenic composition comprising one or more cystatin antigens, wherein the cystatin corresponds to a polypeptide comprising an amino acid sequence which is at least 80% homologous to the amino acid sequence of SEQ ID NO: 1 or SEQ ID NO: 2, and thereby detecting a tick infestation in a subject.

In one embodiment, the methods further comprise the step of monitoring the subject for an inflammatory response or a pain response, wherein an inflammatory response or a pain response indicates a tick infestation.

In another aspect, the invention features a method for the detection of a tick infestation in a subject comprising administering to the subject an immunogenic composition comprising one or more cystatin antigens, and monitoring the subject for an inflammatory response or a pain response, wherein an inflammatory response or a pain response indicates a tick infestation; thereby detecting a tick infestation in a subject.

In another aspect, the invention features a method for the detection of a tick infestation in a subject comprising administering to the subject an immunogenic composition comprising one or more cystatin antigens, where the cystatin corresponds to a polypeptide comprising an amino acid sequence which is at least 80% homologous to the amino acid sequence of SEQ ID NO: 1 or SEQ ID NO: 2, and monitoring the subject for an inflammatory response or a pain response, wherein an inflammatory response or a pain response indicates a tick infestation, thereby detecting a tick infestation in a subject.

In one embodiment, an inflammatory response is indicated by red skin or swollen skin.

In another embodiment, the detection of tick infestation occurs within one hours after tick infestation.

In another particular embodiment, the detection of tick infestation occurs less than 72 hours after tick infestation.

In another embodiment, a pain response at the site of tick infestation is detected by the subject.

In another aspect, the invention features a method of decreasing the ability of a tick to feed on a subject comprising administering to the subject an immunogenic composition comprising one or more cystatin antigens, and thereby decreasing the ability of a tick to feed on a subject.

In still another aspect, the invention features a method of decreasing the ability of a tick to feed on a subject comprising administering to the subject an immunogenic composition comprising one or more cystatin antigens, where the cystatin corresponds to a polypeptide comprising an amino acid sequence which is at least 80% homologous to the amino acid sequence of SEQ ID NO: 1 or SEQ ID NO: 2, and thereby decreasing the ability of a tick to feed on a subject.

In another aspect, the invention features a method for preventing a tick infestation in a subject comprising administering to the subject an immunogenic composition comprising one or more cystatin antigens, thereby preventing a tick infestation in a subject.

In still another aspect, the invention features a method for preventing a tick infestation in a subject comprising administering to the subject an immunogenic composition comprising one or more cystatin antigens, wherein the cystatin corresponds to a polypeptide comprising an amino acid sequence which is at least 80% homologous to the amino acid sequence of SEQ ID NO: 1 or SEQ ID NO: 2, and thereby preventing a tick infestation in a subject.

In one embodiment of any one of the above-mentioned aspects, the tick belongs to the superfamily Ixodoidea. In a related embodiment, the tick is selected from the group consisting of: Ixodes spp, Dermacentor spp, Rhipicephalus spp, Amblyomma spp, Hyalomma spp, Haemaphysalis spp, Boophilus spp, Argas spp, and Ornithodoros spp. In yet another embodiment, the tick transmits a pathogen selected from the group consisting of: Borrelia spp., Anaplasma spp., Theileria spp., Babesia spp., and viruses within the tick-borne encephalitis complex.

In one aspect, the invention features a method for determining a risk for Lyme Disease, Anaplasmosis, East Coast Fever, Babesiosis, or tick-borne Encephalitis in a subject comprising administering to the subject an immunogenic composition comprising one or more cystatin antigens, and monitoring the subject for an inflammatory response or a pain response, wherein an inflammatory response or a pain response indicates a risk for Lyme Disease, Anaplasmosis, East Coast Fever, Babesiosis, or Encephalitis, and thereby determining a risk for Lyme Disease, Anaplasmosis, East Coast Fever, Babesiosis, or tick-borne Encephalitis in a subject.

In still another aspect, the invention features a method for determining a risk for Lyme Disease, Anaplasmosis, East Coast Fever, Babesiosis, or tick-borne Encephalitis in a subject comprising administering to the subject an immunogenic composition comprising one or more cystatin antigens, wherein the cystatin corresponds to a polypeptide comprising an amino acid sequence which is at least 80% homologous to the amino acid sequence of SEQ ID NO: 1 or SEQ ID NO: 2, and monitoring the subject for an inflammatory response or a pain response, wherein an inflammatory response or a pain response indicates a risk for Lyme Disease, Anaplasmosis, East Coast Fever, Babesiosis, or Encephalitis, and thereby determining a risk for Lyme Disease, Anaplasmosis, East Coast Fever, Babesiosis, or tick-borne Encephalitis in a subject.

In one embodiment, the method further comprises the step of treating the subject with a therapeutic or prophylactic agent for Lyme Disease, Anaplasmosis, East Coast Fever, Babesiosis, or tick-borne Encephalitis.

In another aspect, the invention features a method for the prevention of Lyme Disease, Anaplasmosis, East Coast Fever, Babesiosis, or Encephalitis in a subject comprising administering to the subject an immunogenic composition comprising one or more cystatin antigens, and thereby preventing Lyme Disease, Anaplasmosis, East Coast Fever, Babesiosis, or tick-borne Encephalitis in a subject.

In still another aspect, the invention features a method for the prevention of Lyme Disease, Anaplasmosis, East Coast Fever, Babesiosis, or tick-borne Encephalitis in a subject comprising administering to the subject an immunogenic composition comprising one or more cystatin antigens, wherein the cystatin corresponds to a polypeptide comprising an amino acid sequence which is at least 80% homologous to the amino acid sequence of SEQ ID NO: 1 or SEQ ID NO: 2, and thereby preventing Lyme Disease, Anaplasmosis, East Coast Fever, Babesiosis, or tick-borne Encephalitis in a subject.

In one embodiment, the composition further comprises a small molecule inhibitor. In a related embodiment, the small molecule inhibitor is selected from the group consisting of: siRNA, shRNA, DNA aptamers, RNA aptamers, and antisense oligonucleotides. In still another related embodiment, the small molecule inhibitor is an inhibitor of a cystatin.

In another aspect, the invention features a method for the treatment or prevention of a tick infestation in a subject comprising administering to the subject an immunogenic composition comprising one or more cystatin antibodies, or fragments thereof, and thereby treating or preventing a tick infestation in a subject.

In still another aspect, the invention features a method for the treatment or prevention of a tick infestation in a subject comprising administering to the subject an immunogenic composition comprising one or more cystatin antibodies, or fragments thereof, wherein the cystatin corresponds to a polypeptide comprising an amino acid sequence which is at least 80% homologous to the amino acid sequence of SEQ ID NO: 1 or SEQ ID NO: 2, and thereby treating or preventing a tick infestation in a subject.

In another aspect, the invention features a method for reducing the risk of transmission of a tick in a subject comprising administering to the subject an immunogenic composition comprising one or more cystatin antibodies, or fragments thereof, and thereby reducing the risk of transmission of a tick.

In still another aspect, the invention features a method for reducing the risk of transmission of a tick in a subject comprising administering to the subject an immunogenic composition comprising one or more cystatin antibodies, or fragments thereof, wherein the cystatin corresponds to a polypeptide comprising an amino acid sequence which is at least 80% homologous to the amino acid sequence of SEQ ID NO: 1 or SEQ ID NO: 2,and thereby reducing the risk of transmission of a tick.

In another aspect, the invention features a method for treating or preventing Lyme Disease, Anaplasmosis, East Coast Fever, Babesiosis, or tick-borne Encephalitis in a subject comprising administering to the subject an immunogenic composition comprising one or more cystatin antibodies, or fragments thereof, and thereby preventing Lyme Disease, Anaplasmosis, East Coast Fever, Babesiosis, or tick-borne Encephalitis in a subject.

In still another aspect, the invention features a method for treating or preventing Lyme Disease, Anaplasmosis, East Coast Fever, Babesiosis, or tick-borne Encephalitis in a subject comprising administering to the subject an immunogenic composition comprising one or more cystatin antibodies, or fragments thereof, wherein the cystatin corresponds to a polypeptide comprising an amino acid sequence which is at least 80% homologous to the amino acid sequence of SEQ ID NO: 1 or SEQ ID NO: 2, and thereby preventing Lyme Disease, Anaplasmosis, East Coast Fever, Babesiosis, or tick-borne Encephalitis in a subject.

In one embodiment of any one of the above-mentioned aspects, the immunogenic composition further comprises one or more additional antigens.

In a related embodiment, the additional antigens correspond to components isolated or derived from tick saliva. In a further embodiment, the one or more additional antigens modulate host hemostasis. In still another further embodiment, the one or more additional antigens modulate host immunity.

In another embodiment, the additional components isolated or derived from tick saliva are selected from the group consisting of: coagulation modulators, fibrinolysis modulators, angiogenesis modulators, and immune response modulators.

In one embodiment, the coagulation modulator is selected from the group consisting of Factor Xa inhibitors, tissue factor pathway inhibitors, and direct thrombin inhibitors. In a related embodiment, the Factor Xa inhibitor is selected from the group consisting of: TAP, SALP14, and FXa inhibitor (FXaI). In another related embodiment, the direct thrombin inhibitor is selected from the group consisting of: Microphilin, Savignin, Ornithodorin , Madanin 1 and 2 and Variegin. In still another further embodiment, the the immune response modulator is selected from the group consisting of: a complement inhibitor, a T cell inhibitor and a B-cell inhibitor.

In still another particular embodiment, the immune response modulator is Salp15.

In certain embodiments, the immunogenic composition comprising one or more cystatin antigens is administered in nanomolar concentrations. In other embodiments, the immunogenic composition comprising one or more cystatin antigens is administered in picomolar concentrations.

Preferably, in certain embodiments, the concentration is between 50-5000 nM. In further embodiments, the concentration is between 50-100 nM. In other embodiments, the concentration is between 200-400 uM.

In one embodiment, the methods further comprise the step of treatment with an antibiotic.

In another embodiment, the tick belongs to the superfamily Ixodoidea. In a related embodiment, the tick is selected from the group consisting of: Ixodes spp, Dermacentor spp, Rhipicephalus spp, Amblyomma spp, Hyalomma spp, Haemaphysalis spp, Boophilus spp, Argas spp, and Ornithodoros spp. In another related embodiment, the tick transmits pathogens selected from the group consisting of: Borrelia spp., Anaplasma spp., Theileria spp., Babesia spp., and viruses within the tick-borne encephalitis complex.

In another embodiment, the antibody, or fragment thereof, is selected from the group consisting of: monoclonal or polyclonal antibodies.

In a further embodiment, the composition further comprises an adjuvant.

In another further embodiment, the composition is administered by one or more routes selected from the group consisting of: subcutaneous, intradermal, intramuscular, intratumoral injection and transdermal delivery.

In another embodiment, the composition is administered 1, 2, 3, 4, or more times a year.

In another embodiment, the cystatin is selected from Sialostatin L or Sialostatin L2.

In one embodiment of any of the above aspects, the cystatin corresponds to a polypeptide comprising an amino acid sequence which is at least 80% homologous to the amino acid sequence of SEQ ID NO: 1 and SEQ ID NO: 2, respectively.

In still another aspect, the invention features an immunogenic composition comprising one or more cystatin antigens in combination with one or more one or more additional antigens.

In one embodiments, the additional antigens correspond to components isolated or derived from tick saliva. In an further embodiment, the one or more additional antigens modulate host hemostasis. In another related embodiment, the one or more additional antigens modulate host immunity.

In another embodiment, the additional components isolated or derived from tick saliva are selected from the group consisting of: coagulation modulators, fibrinolysis modulators, angiogenesis modulators, and immune response modulators.

In another further embodiment, the cystatin is selected from Sialostatin L or Sialostatin L2.

In yet another further embodiment, the cystatin corresponds to a polypeptide comprising an amino acid sequence which is at least 80% homologous to the amino acid sequence of SEQ ID NO: 1 and SEQ ID NO: 2, respectively.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “adjuvant” is meant to refer to a compound, or combination of compounds which, while not having any specific antigenic effect alone can stimulate or potentiate an immune response. Exemplary adjuvants include, but are not limited to, CpG motifs, LPS, MPL, MF59, RIBI DETOX™, Alum, QS-21, Freund's complete adjuvant, Freund's incomplete adjuvant, MDP, TDM, ISCOMS, Adjuvant 65, Lipovant, TITERMAX, Montanide ISA720, BCG, Levamisole, squalene, Pluronic, TWEEN, or inulin, or protein conjugates (KLH: keyhole limpet hemocyanin for example).

By “administration” or “administering” are meant to include an act of providing a compound or pharmaceutical composition of the invention to a subject in need of treatment.

By “agent” is meant a polypeptide, polynucleotide, or fragment, or analog thereof, small molecule, or other biologically active molecule.

By “antibody” is meant any immunoglobulin, including antibodies and fragments thereof, that binds a specific epitope. The term encompasses polyclonal, monoclonal, and chimeric antibodies (e.g., bispecific antibodies). Exemplary antibody molecules are intact immunoglobulin molecules, substantially intact immunoglobulin molecules, and immunoglobulin molecules including including Fab, Fab′, F(ab′)₂ and F(v) portions.

By “antigen” is meant to refer to any substance that causes the immune system to produce antibodies against it. An antigen may be a foreign substance from the environment such as chemicals, bacteria, viruses, or pollen. An antigen may also be formed within the body, as with bacterial toxins or tissue cells. The term is meant to encompass any antigenic or immunogenic polypeptides including poly-amino acid materials having epitopes or combinations of epitopes, and immunogen-encoding polynucleotides.

By “cystatin” is meant to refer to a family of reversible inhibitors of papain-like cysteine proteases. Cystatins are subdivided into three individual families 1, 2, and 3. An exemplary cystatin is exemplified by GenBank NCBI accession number 22164282.

By a “cystatin protein fragment” is meant a portion of a cystatin protein that has immunomodulatory activity. In certain embodiments, the protein fragment does not have any immunomodulatory action.

By “cystatin nucleic acid molecule” is meant a nucleic acid molecule that encodes a cystatin or biologically active fragment thereof.

By “fragment” is meant a portion of a protein or nucleic acid that is substantially identical to a reference protein or nucleic acid. In some embodiments the portion retains at least 50%, 75%, or 80%, or more preferably 90%, 95%, or even 99% of the biological activity of the reference protein or nucleic acid described herein. In other embodiments, the fragment comprises at least 5, 10, 11, 12, 13, 14, 15, 16, 17, 18, 19, or 20 amino acids of a reference protein or is a nucleic acid molecule encoding such a fragment.

By “homologous” is intended to include a first amino acid or nucleotide sequence which contains a sufficient or minimum number of identical or equivalent amino acid residues or nucleotides, e.g., an amino acid residue which has a similar side chain, to a second amino acid or nucleotide sequence such that the first and second amino acid or nucleotide sequences share common structural domains and/or a common functional activity.

By “infestation” is meant to refer to the bite of one or more than one infected ticks. An infestation can be the presence and attachment of a tick to a subject or an infestation, in certain embodiments, can refer to a subject coming in contact with a tick, but the tick does not remain attached. An infestation may or may not result in a condition or disorder that is caused by a tick, e.g. Lyme disease.

By an “immune” or an “immunogenic response” is meant to include response includes responses that result in at least some level of immunity in the treated subject, where the subject was treated with a composition comprising at least one protein of the present invention.

By “inflammatory response” is meant to refer to the process whereby inflammatory cells are recruited from the blood to lymphoid as well as non-lymphoid tissues via a multifactorial process that involves distinct adhesive and activation steps.

By the phrase “in combination with” is intended to refer to all forms of administration that provide the compounds of the invention together, and can include sequential administration, in any order.

By “polypeptide” is meant any chain of amino acids, regardless of length or post-translational modification.

By “sialostatin” is meant to refer to an active family 2 cystatin. In certain embodiments, a Sialostatin has target specificity directed against cathepsin L and is referred to as “Sialostatin L” or “Sialostatin L2.” In further preferred embodiments, the Sialostatin has anti-inflammatory action.

By “subject” is meant any animal that is susceptible to infestation by a tick. A subject can include, but is not limited to vertebrates, including humans; livestock, such as chickens, turkeys, ostriches, ducks, geese, cattle, pigs, and horses; pets, such as cats, dogs, and horses; and animals that might be held in a zoo.

By “treat,” “treating,” “treatment,” and the like are meant to refer to reducing or ameliorating a disorder and/or symptoms associated therewith. It will be appreciated that, although not precluded, treating a disorder or condition does not require that the disorder, condition or symptoms associated therewith be completely eliminated.

By “tick” is meant to refer to organisms belonging to the superfamily Ixodoidea. Ticks according to the invention can be at any developmental stage e.g. larvae, nymphs, or adults.

### BRIEF DESCRIPTION OF THE DRAWINGS

FIGS. 1A-1C. FIG. 1A shows amino acid (aa) sequence alignment of the two secreted cystatins from Ixodes scapularis. Asterisks and shaded boxes denote conserved, common residues in both proteins. Regions indicated with a line show amino acids that are predicted to play a role in inhibition of cysteine proteases by forming the interaction interface with the active site of the enzyme. Sialostatin L (SEQ ID NO: 1) and Sialostatin L2 (SEQ ID NO: 2). FIG. 1B is a graph illustrating proteolytic enzymes targeted by sialostatin L2. The graph shows percent remaining enzymatic activity. Cathepsins L, V, C, and S are targeted by sialostatin L2. The abscissa represents sialostatin L2 concentration (M) in log10 scale, and the ordinate shows the percentage of remaining enzymatic activity in the presence of sialostatin L2. Each experiment was performed in triplicate. Additional details can be found in Table 1. FIG. 1C is a panel of four graphs showing sialostatin L2 differs in affinity for two common enzymatic targets when compared with sialostatin L. The two inhibitors were allowed to interact with the same amount of enzyme under the same assay conditions. The resulting reduction of enzymatic activity was plotted against the corresponding inhibitor concentration. The abscissa represents inhibitor concentration (M) in log10 scale, and the ordinate shows the percentage of remaining enzymatic activity in the presence of the inhibitor. Each experiment was performed in triplicate. Additional details can be found in Table 1.

FIG. 2A-2C are graphs demonstrating that sialostatin L2 is a tight binding inhibitor for cathepsin L. FIG. 2A shows that a lower inhibitor concentration is necessary for the same percentage of cathepsin L inhibition to be achieved, as the concentration of the enzyme used in the assays decreases from 75 to 12.5 pM. Each experiment was performed in triplicate. The abscissa represents sialostatin L2 concentration (M) in log10 scale, and the ordinate represents the percentage of remaining cathepsin L activity in the presence of sialostatin L2. FIG. 2B shows that the reduction in sialostatin L2 concentration at which 50% inhibition of cathepsin L activity is achieved (IC50) is analogous to the reduction of cathepsin L concentration used in the assay. The abscissa represents (IC50)±SE of triplicates, and the ordinate represents cathepsin L concentration. FIG. 2C shows the relationship of the apparent dissociation constant Ki* to substrate concentration when reactions were initiated by addition of cathepsin L. Values for Ki* were calculated as described in the text. Linear regression of the data yields a Ki of 65.5±23.1 pM (r2=0.992). Each point in the graph is the mean Ki*±SE of four independent experiments.

FIG. 3 is a graph that demonstrates the two sialostatin proteins differ in antigenicity. Female Swiss Webster mice, 6-8 weeks old, were vaccinated as described in Experimental procedures. Plates (96 well) were coated with either sialostatin L or L2 followed by ELISA using pre-immune sera (P.I.S.), sera from vehicle-vaccinated mice (V.V.S.), sera from sialostatin L-vaccinated mice (L.V.S.), or sera from sialostatin L2-vaccinated mice (L2.V.S.). Each group consisted of six mice. The ordinate shows mean milliabsorbance units of the ELISA read (λ=405 nM) for each sample serum±SE. **, statistically significant difference (P<0.001); *, statistically significant difference (P<0.05) in the absorbance read when corresponding sera were tested.

FIG. 4A-4E demonstrates in vivo sialostatin L2 RNAi in the salivary glands of Ixodes scapularis. FIG. 4A shows results of RT-PCR with total RNA prepared from water-injected control salivary glands (Lanes 1, 3, 5, respectively) or sialostatin L2 RNAi salivary glands (Lanes 2, 4, 6, respectively) using sialostatin L2 (Lanes 1, 2), ISAC (Lanes 3, 4), and β-actin (Lanes 5, 6) gene-specific primers for transcript amplification. FIG. 4B is a graph that shows sialostatin L2 RNAi ticks are unable to feed successfully; three experiments were performed on different dates during the active adult tick feeding period using different batches of ticks and New Zealand rabbits. Each experiment was carried out with water-injected control (n=50) and sialostatin L2 dsRNA-injected (n=50) ticks. FIG. 4C is a photograph that illustrated that the percentage of feeding inhibition was calculated by counting dead ticks attached to rabbit ear (arrows) during the first 24-48 h of infestation. FIG. 4D is a graph that shows partially fed female adult ticks were pulled from rabbit on days 4, 5 and 7 (n=10 and n=10, respectively) and weighed during each experiment. The ordinate shows the average tick weight (in mg) of three replicate experiments; *, statistically significant difference (P<0.05). FIG. 4E is a photograph that shows fully engorged female adult ticks, representing the control and the experimental group that dropped off and were kept for egg mass recovery.

FIG. 5A-5C demonstrates the immunomodulatory role of sialostatin L2 during tick feeding on vertebrate host. FIG. 5A is a graph depicting the results of an experiment where naïve adult female ticks (n=50) were allowed to feed on rabbits previously exposed to sialostatin L2 RNAi or water-injected control ticks, respectively. Each experiment was carried out three times and the percentage of dead, de-attached, or fed-to-repletion ticks calculated for experimental and control groups; **, statistically significant difference (P<0.001); *, statistically significant difference (P<0.05). FIG. 5B is a photograph illustrating that naïve female adult ticks attached but died in the first 24 h of infestation in rabbits previously exposed to sialostatin L2 RNAi ticks. In this characteristic photo from the ear of such a rabbit, arrows with asterisks indicate swollen skin; black arrows are dead attached ticks; the double arrow is a site of profound inflammation. FIG. 5C is a photo of naïve female adult ticks that managed to feed to repletion when attached to rabbits previously infested with water-injected control ticks.

FIG. 6A-6E demonstrates the effect of the administration of a Sialostatin L2 immunogenic composition on on tick feeding in Guinea Pigs. FIG. 6A shows the percentage of Ixodes scapularis nymphs that failed to feed on the control and the sialostatin L2 vaccinated group. FIG. 6B shows a characteristic photo of the size of engorged Ixodes scapularis nymphs fed for 96 hours in control and sialostatin L2 vaccinated Guinea Pigs. FIG. 6C shows the average repletion weight of the nymphs fed in the control and the sialostatin L2 vaccinated Guinea Pigs by the end of the experiment. FIG. 6D provides the same information but additionally shows the weight of each individual nymph as a dot in the graph. FIG. 6E shows the percentage of engorged Ixodes scapularis nymphs that exceeded or weighed lesser a certain weight shown in the abscissa. **, statistically significant difference (P<0.001); *, statistically significant difference (P<0.05).

FIG. 7A-7E also demonstrates the effect of the administration of a Sialostatin L2 immunogenic composition on tick feeding in Guinea Pigs. 7A-7C demonstrate apparent inflammation signs in the attachment sites of nymphs in sialostatin L2-vaccinated Guinea Pigs. FIG. 7C shows that signs of inflammation developed in the nymphal attachment sites of sialostatin L2 vaccinated animals. Apparent signs of inflammation (redness and edema formation) could be detected in the attachment sites of some nymphs 72 h post infestation in the vaccine group. Their size comparison indicates that some of them were affected in their feeding ability (upper tick), while this was not always the case (lower tick). FIG. 7D-7E show the sialostatin L2 vaccinated and control groups 96hours post tick attachment.

FIG. 8 is a graph that shows higher early rejection was observed for the nymphs attached on sialostatin L2 vaccinated animals. The graph shows that variation in the antisialostatin L2 IgG titer was detected among vaccinated animals (#1-4) 2 wks after their last vaccination. The mean titer of each animal (estimated from triplicates and divided by 104) is represented (red bars in the graph), as well as the SE in the titer estimation. The percentage of ticks rejected prematurely (within the first 72 hours of attachment) for each animal is represented with green bars. These nymphs were found detached from the host and their mean body weight was similar to that of ticks prior to host attachment. Four animals were used in both groups.

FIG. 9A-9C shows impaired feeding of I. scapularis nymphs when attached on sialostatin L2 vaccinated animals. The graph in panel A shows increased rejection was observed for the ticks attached to sialostatin L2 vaccinated guinea pigs. The bar represents the mean percentage of early tick rejection (see results) from the control and the experimental group, while the lines represent the standard error of the mean. Each group consisted of four animals. The asterisk denotes a statistically significant difference between the means of the two experimental groups (P<0.05). The grah in panel B shows that there was a statistically significant delay in drop off of ticks attached in the vaccine group compared with those in the control group between 4 and 6 days after initial attachment, as shown in the graph. The photo shows the difference in size of the ticks recovered from the two experimental groups 4 days post initial attachment. Panel C is a graph that shows the distribution of weight of nymphs recovered from the control and the vaccine groups. A statistically significant difference in mean nymphal weight is seen (1.9 mg for the nymphs attached to the vaccine group versus 2.8 mg for those attached to the control group, represented with a line in the distribution graph). The asterisk denotes a statistically significant difference between the means of the two experimental groups (P<0.05).

FIG. 10 is a graph showing that higher reduction of tick feeding ability was observed for the animals displaying the higher antisialostatin L2 titer The mean titer of each animal (estimated from triplicates and divided by 104) is represented (solid bars in the graph), as well as the SE in the titer estimation, while the average weight (in grams multiplied with 104) per tick initially attached in each animal is represented with shadowed bars. Four animals were used in both groups.

FIGS. 11A and 11B are graphs that show the mechanism underlying the observed feeding impairment of I. scapularis nymphs. In panel A a statistically significant inhibition of sialostatin L2 action could be observed in in vitro assays for cathepsin L inhibition (9) upon addition of 1 or 5 μl of antisialostatin L2 purified IgGs (but not when adding purified IgGs from control animals) in 50 μl of reaction mix. Three different concentrations of sialostatin L2 were tested in the sera inhibition assays (5 nM, 2 nM, and 1 nM) in triplicate. In panel B, a boost in the mean titer of the vaccinated guinea pigs was observed two wks post tick infestation. The bar represents the mean from the sialostatin. L2 vaccinated guinea pigs before and after exposure to ticks, while the lines represent the standard error of the mean. The group consisted of four animals. The asterisk denotes a statistically significant difference between the mean antisialostatin L2 titer before and after exposure to ticks (P<0.05).

### DETAILED DESCRIPTION OF THE INVENTION

The invention generally features methods for the prevention and detection of a tick infestation. The present invention also features methods for decreasing the ability of a tick to feed on a subject. The present invention is based in part on the finding of an active cystatin in the saliva of the tick I. scapularis that results in a transcription-regulated boost of saliva inhibitory activity against a number of vertebrate papain-like cysteine proteases during blood feeding. The invention uncovers a role of the targeted enzymes in vertebrate immunity, and describes that host immunomodulation is implicated in the deleterious phenotype of silenced ticks, making the cystatins attractive targets for development of anti-tick immunomodulatory compositions.

Additionally, the invention describes the potential for development of a multicomponent vaccine that will protect against tick bites and the pathogens they transmit.

Accordingly, the invention provides methods for preventing and detecting tick cystatin salivary secretion associated diseases and disorders.

### Cystatins

Cystatins are present in vertebrates, invertebrates, plants, and protozoa, and all of them form tight, equimolar, and reversible inhibitory complexes with papain-like cysteine proteases. Cysteine proteases have traditionally been considered as mediators of the terminal bulk proteolysis inside the lysosome. As a result, the vertebrate cystatins have been the focus of research as the guardians or regulators that ensure protection of cells and tissues against the undesirable scission of peptide bonds and damage that could be caused when cysteine proteases are released outside their normal compartment.

Since the first description of chicken egg white cystatin in the late 1960s (46), a body of information has been accumulated for this superfamily of proteins present in vertebrates, invertebrates, plants, and protozoa. Cystatins are further subdivided into three individual families, namely 1, 2, and 3. Family 1 members (also known as stefins) are cytosolic molecules with neither disulfide bonds nor carbohydrates. Family 2 contains all of the secreted cystatins that are mainly found in biologic fluids; they form two disulfide bridges, and they do not bear sugars. In contrast to the members of the previous two families, which possess a single cystatin-like domain and display low molecular mass (11-14 kDa), each family 3 cystatin (also known as kininogens) is made of several cystatin modules, and thus being relatively larger molecules (60-120 kDa) (47).

Structural studies of various cystatins show that they display a wedge-shaped interface that binds to the active site of their target proteases (17). This interface consists of three typical segments (18) the N-terminal domain located around a conserved G (PI segment); a hairpin loop located around the conserved sequence QXVXG (PII segment); and a second hairpin loop located around a conserved PW dipeptide (PIII segment).

Recently, two secreted cystatins from the soft tick Ornithodoros Moubata have been described. Soft ticks feed rapidly, so their cystatins are shown to play a role in midgut physiology rather than in salivary glands. Both soft tick cystatins display the PW motif in their PIII segment and inhibit cathepsins B and H. The same holds true for a secreted cystatin from the hard tick Haemaphysalis longicornis that plays a role in tick midgut physiology/innate immunity (21) but not in salivary glands. There are several other amino acid (aa) differences throughout those proteins, however another salivary cystatin from the hard tick Amblyomma americanum has the NL substitution in PIII segment, and RNAi silenced ticks displayed reduced ability to feed successfully in rabbits (10). Although biochemical characterization of this protein is still lacking, as is that of transcriptional regulation of its gene, it is possible that divergence of the sequence in the PIII segment of salivary hard tick cystatins, and the resulting lack of inhibition for cathepsins B and H is a contributor to the conserved role of those molecules in hard tick feeding success in the vertebrate host.

Previously, a cystatin, sialostatin L, was described because of its affinity for cathepsin L (9). It was further shown that tick saliva displays inhibitory activity against cathepsin L in vitro, that could be partially attributed to the presence of sialostatin L. Recently, a second cystatin has been described, which is named sialostatin L2 to emphasize its inhibitory activity against cathepsin L (74). The amino acid sequences sialostatins L and L2, corresponding to SEQ ID NO: 1 and SEQ ID NO: 2, accordingly, are shown below:

Sialostatin L
(SEQ ID NO: 1)
MTGVFGGYSERANHQANPEFLNLAHYATSTWSAQQPGKTHFDTVAEVV
KVETQVVAGTNYRLTLKVAESTCELTSTYNKD
TCLPKADAAHRTCTTVVFENLQGDKSVSPFECEAA
Sialostatin L2
(SEQ ID NO: 2)
MELALRGGYRERSNQDDPEYLELAHYATSTWSAQQPGKTHFDTVVEVL
KVETQ
TVAGTNYRLTLKVAESTCELTSTYNKDTCQANANAAQRTCTTVIYRNL
QGEKSISSFECAAA

The sialostatin L and L2 proteins have a leader sequence which is characteristic of members of the larger cystatin family. In preferred embodiments, the leader sequence, corresponding to residues MTSTFALVLLLGGMAVCVA and MTSSLALVLLFGGAAVCA respectively, is not included in the sialostatin proteins of the instant invention.

Other than high amino acid identity and similar affinity for cathepsin L, the two cystatins are not equally potent in inhibition of other target enzymes, and further the two cystatins differ in antigenicity. Moreover, as described herein, there are major differences in transcript abundance of the two sialostatins during tick infestation: sialostatin L2 transcripts greatly accumulate in the salivary glands as feeding to the host progresses, while sialostatin L transcripts slightly decrease at the same time.

### Cathepsins

Cathepsins are part of the cysteine protease superfamily. Cysteine proteases are proteases which are distinguished by the presence of a cysteine residue in the active site of the protease which plays a critical role in the catalytic process.

Cathepsins are widely distributed and differentially expressed among tissues. These enzymes have a role in processes that involve proteolysis and turnover of specific proteins and tissues in local microenvironments. Cathepsins also initiate proteolytic cascades by proenzyme activation, participate in the expression of functional MHC class II molecules which bind to antigenic peptides, and process antigen in antigen-presenting cells. The various members of this family are differentially expressed, and some forms of cathepsins are closely associated with monocytes, macrophages, and other cells of the immune system. The secreted forms of several members of this family function in tissue remodeling through degradation of collagen, laminin, elastin, and other structural proteins and are implicated in inflammation associated with immunological response and in metastasis.

In the era of the human genome, it has been shown that the group of human papain-like cysteine proteases numbers more than 11 members (26). Currently known forms of cathepsins include cathepsin B, C, F, H, J, K, L, M, O, Q, R, S, T, U, V, W and Z. Cathepsin L, a target of the sialostatins, is unique among cathepsins by having an important extracellular function. Up to 40% of the cathepsin L proenzyme from fibroblasts is secreted and shows catalytic activity even in the absence of further maturation processing. Cathepsin L is more efficient in the degradation of protein substrates than other members of the same family and is more effective in the hydrolysis of extracellular matrix proteins, such as collagen and elastin, even when compared with collagenases and neutrophilic elastase, which are better known for their activity on these substrates (59, 60).

Within the last decade, a series of studies have expanded the understanding of cysteine proteases and showed their much more expanded role in certain aspects of vertebrate biology (36). Besides their implication in antigen presentation (37) and immune system development (38), they are also involved in epidermal homeostasis (39), neovascularization (40), extracellular matrix degradation and neutrophil chemotaxis during inflammation (41, 42), and apoptosis (43). Moreover, cysteine proteases have been associated with a number of pathologic events including, but not limited to, the proliferation of malignant cells and their subsequent invasion into healthy tissues during metastasis (44, 45), rheumatoid arthritis, osteoarthritis, Alzheimer disease (AD), multiple sclerosis, and muscular dystrophy (56,58).

Secreted cystatins seem to have access to intracellular compartments (61), and as a result, the cystatins, in particular sialostatin L or L2, besides having specificity for cathepsins L, could affect the activity of additional enzymes by blocking proteolytic cascades that take place during the maturation of their proenzymes. More specifically, cathepsin L and S are responsible for the removal of the inhibitory pro-region of procathepsin C (62), and in the presence of sialostatin L or L2, cathepsin L activity is inhibited. Sialostatin inhibition of cathepsin C should also prevent the activation of granule serine proteases in CTL and natural killer cells (granzymes A and B), mast cells (tryptase, proteinase 3, and chymase), and neutrophils (cathepsin G and elastase), because the N-terminal dipeptides of their proenzymes would not be removed (63-65). Indeed, it could result in prevention of cathepsin B maturation, since the trimming of the N-terminal extensions of cathepsin B propeptide is no longer possible (66). Additionally, cathepsin L inhibition could affect cathepsin D processing. Thus, it is possible that sialostatin L and sialostatin L2 target fundamental enzymes controlling the activation of proteolytic cascades in both the extracellular and intracellular compartments (74).

### Ticks and Disease Transmission

Among the differences that make a relationship between two organisms parasitic rather than symbiotic is the lack of mutual benefit; the parasite manages to continuously receive valuable resources from the host without returning this favor; in addition, sometimes it triggers catastrophic conditions to the host such as disease transmission. Hard ticks can be considered an exemplary case of efficient ectoparasites that are able to suck blood—a rich source of nutrients—from their vertebrate host(s) for several days (1). If the ‘blood donor’ is aware of the tick attack to the skin/blood circulation, given that a tick cannot fly, rejection could be the best scenario and death the worst for the arthropod. Consequently, ticks have developed a series of mechanisms to gain undisturbed access to their nutritious meal, including saliva injection in biting sites (2). Tick salivary glands regulate water and ion excretion by saliva secretion that in addition reduces the volume of the blood bolus in the tick digestive tract as feeding progresses. Furthermore, they deliver a repertoire of pharmacologic compounds in the site of infestation that affects among other things, hemostasis and host immunity, thus facilitating the completion of a good quality meal for the tick(3). Unluckily for the host, saliva has also been shown to enhance tick vector competence, e.g., its capability to transmit pathogens (4).

Ticks according to the invention belong to the superfamily Ixodoidea, the superfamily of the suborder Ixodides, which includes the families Argasidae and Ixodidae. Ixodidae includes the genera Amblyomma, Anocentor, Aponomma, Boophilus, Dermacentor, Haemaphysalis, Hyalomma, Ixodes, Margaropus, Rhipicentor, and Rhipicephalus. In certain embodiments of the invention, the tick is selected from the group consisting of Ixodes spp, Dermacentor spp, Rhipicephalus spp, Amblyomma spp, Hyalomma spp, Haemaphysalis spp, Argas spp, Ornithodoros spp, and Boophilus spp. Ticks are vectors of a number of diseases and disorders, some of which can be debilitating or life-threatening. Exemplary pathogens transmitted by ticks include, but are not limited to, Borrelia spp., Anaplasma spp., Theileria spp., Babesia spp., and viruses within the tick-borne encephalitis complex. Accordingly, the pathogen can cause a disease or disorder in the subject including, but not limited to Lyme Disease, Anaplasmosis, East Coast Fever, Babesiosis, or tick-borne Encephalitis.

Lyme Disease

Lyme disease is the most prevalent vector-borne disease of humans in the United States and is transmitted by the bite of Ixodes ticks. Infection is caused by the bacterium Borrelia burgdorferi resulting in an illness affecting various organ systems of the body. The clinical implications of Lyme disease can be seen in dermatologic, neurologic and rheumatologic manifestations.

In Lyme disease two stages of the disease, acute and chronic, are considered. These stages may occur separately or may overlap. Neurological disorders (such as Bell's palsy, meningitis, encephalitis), cardiovascular cardiac arrhythmia, and disorders of the musculoskeletal system (migrating pain in muscles, tendons or joints) are possible in Lyme disease. If left untreated, the patient may acquire chronic lyme borreliosis.

Currently, Lyme Disease is treated with antibiotics. However, such treatment is not always successful in clearing the infection. Treatment is often delayed due to improper diagnosis with the deleterious effect that the infection proceeds to a chronic condition, where treatment with antibiotics is often not useful. One of the factors contributing to delayed treatment is the lack of effective diagnostic tools.

Babesiosis

Babesiosis is a potentially severe, and sometimes rapidly fatal, tick-borne illness caused by a protozoan parasite that infects and destroys the red blood cells. Babesia microti appears to be responsible for the majority of cases of human babesiosis in the United States. It is the most common species in the eastern and Midwestern U.S. where most cases occur. Additional types of Babesia that have been associated with human disease in limited areas of the U.S., but that have not yet been designated as distinct species, are currently known only as Babesia isolate type WA1 parasites (detected on the West Coast) and Babesia isolate type MO1 (detected in Missouri). Babesia divergens is the most common species in Europe. Other Babesia species cause illness in animals.

Based on serologic studies, most infections appear to be asymptomatic. Manifestations of symptomatic disease include fever, headache, chills, sweating, muscle aches (myalgias), fatigue, nausea, vomiting, enlarged spleen and liver (sometimes resulting in jaundice), and hemolytic anemia (anemia due to the destruction of red blood cells). Symptoms usually occur 1 to 4 weeks following an infective tick bite, and can last for several days, weeks, or months. The disease is more severe, and sometimes fatal, in patients who are immunosuppressed lack a healthy spleen, or who are elderly.

Because of the difficulty in diagnosing and treating tick-borne illnesses, the methods described herein are advantageously useful to detect a tick infestation soon after exposure to the tick.

### Methods

Without being bound by theory, the present invention is based on the observation that in the tick, there was an apparent duplication event of cystatin genes in the genome that resulted in a transcription-regulated boost of saliva inhibitory activity against a conserved and relatively limited number of vertebrate papain-like cysteine proteases during blood feeding. As a result, reducing the in vivo activity of cystatin, especially sialostatin L or L2, can be employed according to the present invention to prevent or detect tick infestation. Further, reducing the in vivo activity of cystatin can be used to decrease the ability of a tick to feed on a subject. In effect, decreasing the ability of a tick to feed on a subject will reduce the tick population, and serve as a means to control the tick population.

Due to their small size and difficulty of detection, Ixodes scapularis nymphs are the key vector stage implicated in Lyme transmission in disease endemic regions of the US. An alternative or complementary component of an integrated approach against ticks and/or the pathogens they transmit is the development of anti tick vaccines. This idea is supported by the finding that certain vertebrate hosts (e.g. guinea pigs) develop tick hypersensitivity upon repeated exposure to ticks, preventing ticks from taking a blood meal. This anti tick immunity can, upon tick re-exposure, prevent Borrelia transmission, as well (Nazario, S., S. Das, A. M. de Silva, K. Deponte, N. Marcantonio, J. F. Anderson, D. Fish, E. Fikrig, and F. S. Kantor. 1998. Prevention of Borrelia burgdorferi transmission in guinea pigs by tick immunity. Am J Trop Med Hyg 58:780-785).

Included in the invention are methods for the detection of a tick infestation in a subject comprising administering to the subject an immunogenic composition comprising one or more cystatin antigens, and thereby detecting a tick infestation in a subject.

The method can further include the step of detection.

The detection may be a visual detection, for example of redness or inflammation at the site of the tick bite.

In certain examples, the cystatin corresponds to a polypeptide comprising an amino acid sequence which is at least 80%, 85%, 90%, 95%, 96%, 97%, 98%, 99% or more homologous to the amino acid sequence of SEQ ID NO: 1 or SEQ ID NO: 2.

The methods of the invention may include administering to a subject an immunogenic composition comprising one or more cystatin antigens, in combination with one or more immunogenic components, for example one or more additional antigens. In preferred embodiments of the invention, the one or more additional antigens are isolated or derived from tick saliva.

Work on the transcriptome and proteome of I. scapularis glands (8) has revealed numerous components of its saliva and, more importantly, their potential pharmacologic action on host coagulation (82; 83), fibrinolysis (84), immunity (9; 76; 77; 86), and angiogenesis (87).

This action not only facilitates tick attachment to the vertebrate host and acquisition of the blood meal, but also creates a tick/vertebrate host interface advantageous for pathogen transmission. Therefore, vertebrate host immunity that blocks the action of tick salivary constituents, for example the immunogenic compositions as described herein, have the potential to affect tick feeding ability and transmission of tick borne pathogens.

Accordingly, the invention features immunogenic compositions, e.g. immunogenic compositions comprising one or more cystatin antigens as described herein, further comprising one or more additional antigens.

In preferred embodiments of the invention, the additional antigens correspond to components isolated or derived from, or antigens expressed in, tick saliva.

A catalogue of transcripts that encode for proteins secreted from I. scapularis salivary glands is described by Ribiero J M C et al. (Insect Biochemistry and Molecular Biology 36.2006.111-129), incorporated by reference in its entirety herein. A list of proteins whose activity that has been characterized from tick salivary glands is described by Hovius J W R et al. (PLOS Medicine 2008. February Vol. 5. Issue 2. e43), incorporated by reference in its entirety herein.

For example, the one or more additional antigens may modulate host hemostasis or may modulate host immunity.

More specifically, the additional components isolated or derived from tick saliva can be selected from, but not limited to, the group consisting of: coagulation modulators, fibrinolysis modulators, angiogenesis modulators, and immune response modulators.

Coagulation modulators and fibrinolysis modulators can more generally be components that affect the hemostatic response. The hemostatic response enables mammals to control blood loss during vascular injury. Platelets adhere to macromolecules in exposed subendothelial tissue and aggregate to form a hemostatic plug, while local activation of plasma coagulation factors leads to generation of a fibrin clot that reinforces the platelet aggregate. Tick feeding is hampered by the hemostatic response of the host. Therefore tick saliva contains an extensive selection of molecules that counteract coagulation, enhance fibrinolysis, and inhibit platelet aggregation (Maritz-Olivier C, Stutzer C, Jongejan F, Neitz A W, Gaspar A R (2007) Tick anti-hemostatics: targets for future vaccines and therapeutics. Trends Parasitol 23: 397-407).

Exemplary anticoagulants can be Faxtor Xa inhibitors, tissue factor pathway inhibitors, or direct thrombin inhibitors. Factor Xa inhibitors include, but are not limited to, TAP (Accession No.GI1421459), Salp14 (Accession No. AAK97824), FXa inhibitor (FXaI) (Accession No. AAN76827). Tissue factor pathway inhibitors include, but are not limited to: Ixolaris (Accession No. AAK83022) and Penthalaris (Accession No. AAM93638). Direct thrombin inhibitors include, but are not limited to, Microphilin, Savignin (Accession No. AAL37210), Ornithodorin (Accession No. AAP04349, AAP04350), Variegin. Complement inhibitors include, but are not limited to, OMCI (Accession No. AAT65682), Isac (Accession No. AAF81253), IRAC 1 and 2 Accession Nos. AAX63389, AAX63390), and Salp20 (Accession No. AAK97820). T cell inhibitors include, but are not limited to Salp15 (Accession No. AAK97817(I.scapularis), Accession No. ABU93613 (I. ricinus)), IL-2 binding protein, Iris Accession No CAB55818) and Sialostatin L (Accession No. GI22164282).

Immunosuppressors include, but are not limited to, complement inhibitors, T cell inhibitors and B cell inhibitors.

In a particular embodiment, the coagulation modulator is a complement inhibitor, a tissue factor pathway inhibitor (TFPI), an angiogenesis modulator or an immune response modulator. For example, Salp15 is a tick salivary immunomodulator, and has been described during tick Lyme disease transmission (Ramamoorthi, N., S. Narasimhan, U. Pal, F. Bao, X. F. Yang, D. Fish, J. Anguita, M. V. Norgard, F. S. Kantor, J. F. Anderson, R. A. Koski, and E. Fikrig. 2005. The Lyme disease agent exploits a tick protein to infect the mammalian host. Nature 436:573-577).

Many of the active salivary glands effectors exert their biological action at picomolar to nanomolar concentrations. This can also be the case for sialostatins. Accordingly, such low amount of protein that is able to effect a function enables the salivary gland effectors, including sialostatin, to go unnoticed from vertebrate immune system upon tick infestation. Accordingly, the salivary glad effectors may be called “silent effectors” or “silent antigens.”

In certain preferred embodiments of the invention, in order to immunize/sensitize the animals a range of 0.5 to 100 ug of protein, preferably in 50 to 100 ul of buffer/formulation is used. Such formulation results in a protein concentration (dependent upon the molecular weight (MW) of the protein) between about 50-100 nM to 200-400 uM (low nM to low mM) of protein in the immunogenic formulations.

The methods of detecting a tick infestation in a subject comprise, in certain examples, monitoring the subject for an inflammatory response or a pain response, where an inflammatory response or a pain response indicates a tick infestation. The inflammatory response or the pain response occurs at the site of tick infestation, or the site of tick attachment to the subject. By “inflammatory response” is meant to refer to the process whereby inflammatory cells are recruited from the blood to lymphoid as well as non-lymphoid tissues via a multifactorial process that involves distinct adhesive and activation steps. The inflammatory response may be a local cutaneous inflammatory response to tick attachment, and may be indicated by red skin or swollen skin. A local inflammatory is expected to reduce the ability of tick-borne pathogens to be transmitted to the subjects, by creating a ‘rather unfriendly’ environment for the pathogens in the very initial steps of their transmission.

A pain response is determined by the subject and is in response to tick attachment. Inflammatory or pain responses may occur within 1, 2, 3, 4, 8, 10, 15, 20, 25, 30, 40, 48 or more hours following tick infestation.

The methods of the invention also feature decreasing the ability of a tick to feed on a subject comprising administering to the subject an immunogenic composition comprising one or more cystatin antigens, and thereby decreasing the ability of a tick to feed on a subject. In certain examples, the cystatin corresponds to a polypeptide comprising an amino acid sequence which is at least 80%, 85%, 90%, 95%, 96%, 97%, 98%, 99% or more homologous to the amino acid sequence of SEQ ID NO: 1 or SEQ ID NO: 2.

Advantageously, the methods of decreasing the ability of a tick to feed on a subject will also function to reduce the tick population. Thus, the method has further use to control tick population, by administration of the immunogenic composition to a subject population in order to control the tick population by reducing its ability to feed.

The invention also features methods for preventing a tick infestation in a subject comprising administering to the subject an immunogenic composition comprising one or more cystatin antigens and thereby preventing a tick infestation in a subject. In certain examples, the cystatin corresponds to a polypeptide comprising an amino acid sequence which is at least 80%, 85%, 90%, 95%, 96%, 97%, 98%, 99% or more homologous to the amino acid sequence of SEQ ID NO: 1 or SEQ ID NO: 2.

The methods of the invention have use for determining a subject's risk for or for preventing Lyme Disease, Anaplasmosis, East Coast Fever, Babesiosis, or tick-borne Encephalitis. The methods comprise administering to the subject an immunogenic composition comprising one or more cystatin antigens and monitoring the subject for an inflammatory response or a pain response, wherein an inflammatory response or a pain response indicates a risk for Lyme Disease, Anaplasmosis, East Coast Fever, Babesiosis, or Encephalitis and thereby determining a risk for Lyme Disease, Anaplasmosis, East Coast Fever, Babesiosis, or tick-borne Encephalitis in a subject. In certain examples, the cystatin corresponds to a polypeptide comprising an amino acid sequence which is at least 80%, 85%, 90%, 95%, 96%, 97%, 98%, 99% or more homologous to the amino acid sequence of SEQ ID NO: 1 or SEQ ID NO: 2.

After a subject's risk is determined, it may be desirable to treat the subject with a therapeutic or prophylactic agent for Lyme Disease, Anaplasmosis, East Coast Fever, Babesiosis, or tick-borne Encephalitis. Doxycycline or Amoxicillin or Atovaquone plus Azithromycin are some examples of such a treatment.

In any of the methods of the invention as described herein the immunogenic compositions that are administered may further comprise a small molecule inhibitor. The small molecule inhibitor may be selected from, but not limited to, siRNA, shRNA, DNA aptamers, RNA aptamers, and antisense oligonucleotides. In preferred embodiments, the small molecule inhibitor is an inhibitor of a cystatin. Small molecule inhibitors are described in detail below.

Other methods of the invention feature prevention of a tick infestation in a subject or reduction in the risk of transmission of a tick in a subject, comprising administering to the subject an immunogenic composition comprising one or more cystatin antibodies, or fragments thereof and thereby treating or preventing a tick infestation in a subject. In certain examples, the cystatin corresponds to a polypeptide comprising an amino acid sequence which is at least 80%, 85%, 90%, 95%, 96%, 97%, 98%, 99% or more homologous to the amino acid sequence of SEQ ID NO: 1 or SEQ ID NO: 2.

The methods feature treating or preventing Lyme Disease, Anaplasmosis, East Coast Fever, Babesiosis, or tick-borne Encephalitis in a subject comprising administering to the subject.an immunogenic composition comprising one or more cystatin antibodies, or fragments thereof, and thereby preventing Lyme Disease, Anaplasmosis, East Coast Fever, Babesiosis, or tick-borne Encephalitis in a subject. In certain examples, the cystatin corresponds to a polypeptide comprising an amino acid sequence which is at least 80%, 85%, 90%, 95%, 96%, 97%, 98%, 99% or more homologous to the amino acid sequence of SEQ ID NO: 1 or SEQ ID NO: 2.

Antibodies can be monoclonal or polyclonal antibodies, and are described in detail below.

### Antibodies

The methods of the invention contemplate antibody-based compositions. Accordingly, the instant invention features administering to the subject an immunogenic composition comprising one or more cystatin antibodies, or fragments thereof. In certain embodiments, the immunogenic composition comprises one or more cystatin antibodies, or fragments thereof, where the cystatin corresponds to a polypeptide comprising an amino acid sequence which is at least 80% homologous to the amino acid sequence of SEQ ID NO: 1 or SEQ ID NO: 2.

The antibody based compositions can be used in methods for the treatment or prevention of a tick infestation, methods for reducing the risk of transmission of a tick in a subject, or in methods for treating or preventing Lyme Disease, Anaplasmosis, East Coast Fever, Babesiosis, or tick-borne Encephalitis in a subject.

Antibodies that are both specific for cystatin protein and interfere with its activity may be used to inhibit cystatin function. Where desirable, antibodies specific for mutant cystatin protein may also be used. Such antibodies may be generated using standard techniques (briefly described below) against cystatins themselves or against peptides corresponding to portions of cystatins. The antibodies include but are not limited to polyclonal, monoclonal, Fab fragments, single chain antibodies, chimeric antibodies, etc.

Antibodies of use according to the methods of the invention may include, but are not limited to polyclonal antibodies, monoclonal antibodies (mAbs), humanized or chimeric antibodies, single chain antibodies, Fab fragments, F(ab′)2 fragments, fragments produced by a FAb expression library, anti-idiotypic (anti-Id) antibodies, and epitope-binding fragments of any of the above.

For the production of antibodies to cystatin, various host animals may be immunized by injection with a cystatin protein, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a cystatin gene product, or an antigenic functional derivative thereof. For the production of polyclonal antibodies, host animals such as those described above, may be immunized by injection with a cystatin gene product supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein (1975) Nature 256:495-497; and U.S. Pat. No. 4,376,110, the human B-cell hybridoma technique (Kosbor et al. (1983) Immunology Today 4:72; Cole et al. (1983) Proc. Natl. Acad. Sci. USA 80:2026-2030, and the EBV-hybridoma technique (Cole et al. (1985) Monoclonal Antibodies And Cancer Therapy, Alan R. Liss, Inc., pp. 77-96). Such antibodies may be of any immunoglobulin class including IgG, IgM, IgE, IgA, IgD and any subclass thereof.

In addition, techniques developed for the production of “chimeric antibodies” or “humanized antibodies” may be utilized to modify mouse monoclonal antibodies to reduce immunogenicity of non-human antibodies. Morrison et al. (1984) Proc. Natl. Acad. Sci. 81:6851-6855; Neuberger et al. (1984) Nature, 312:604-608; Takeda et al. (1985) Nature, 314:452-454. Such antibodies are generated by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird (1988) Science 242:423-426; Huston et al. (1988) Proc. Natl. Acad. Sci. USA 85:5879-5883; and Ward et al. (1989) Nature 334:544-546) can be adapted to produce single chain antibodies. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments may include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al. (1989) Science 246:1275-1281) to allow rapid and easy identification of monoclonal Fab fragments with the desired specificity.

Lipofectin or liposomes may be used to deliver the antibody or a fragment of the Fab region which binds to the target gene product epitope into cells. Where fragments of the antibody are used, the smallest inhibitory fragment which binds to the target protein's binding domain is preferred. For example, peptides having an amino acid sequence corresponding to the domain of the variable region of the antibody that binds to the cystatin may be used. Such peptides may be synthesized chemically or produced via recombinant DNA technology using methods well known in the art.

### Small Molecules

Small molecules may also be used to inhibit cystatins. Accordingly, in certain embodiments of the invention, the method further comprises administering a composition that further comprises a small molecule inhibitor. Accordingly, the small molecule inhibitor can be, but is not limited to, siRNA, shRNA, DNA aptamers, RNA aptamers, and antisense oligonucleotides.

The small molecule inhibitor may have higher selectivity toward a particular form of cystatin than other forms of cystatin (e.g. sialostatin L or L2, or cystatin with or without the leader sequence). In preferred embodiments, a small molecule inhibitor is preferably a stronger inhibitor of sialostatin L or L2 than other cystatins. In one variation, the small molecule inhibitor is preferably a stronger inhibitor of sialostatin L2 than any other sialostatin. For example, an agent may be at least a 10 times, 100 times, or 1000 times stronger inhibitor of sialostatin L2 than L.

Nucleic acid-based agents such as antisense molecules and ribozymes can be utilized to target both the introns and exons of the cystatin genes as well as at the RNA level to inhibit gene expression thereof, thereby inhibiting the activity of the targeted cystatin. Techniques for the production and use of such molecules are well known to those of skill in the art and are described briefly herein.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

In vitro studies may be performed to quantify the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control-oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (See, e.g., Letsinger (1989) Proc. Natl. Acad. Sci. U.S.A. 86:6553-6556) or the blood-brain barrier (see, e.g., PCT Publication No. WO89/10134, published Apr. 25, 1988), hybridization-triggered cleavage agents. See, e.g., Krol (1988) BioTechniques 6:958-976 or intercalating agents. See, e.g., Zon (1988) Pharm. Res. 5:539-549. The oligonucleotide may be conjugated to another molecule, e.g., a peptide, hybridization triggered cross-linking agent, transport agent, hybridization-triggered cleavage agent, etc.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore an alternate approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Examples of viral vector include, but are not limited to viral vectors based on recombinant virus, such as modified or recombinant retrovirus, adenovirus, adeno-associated viruses, vaccinia virus, and herpes simplex virus.

Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon (1981) Nature 290:304-310), the promoter contained in the. 3′ long terminal repeat of Rous sarcoma virus (Yamamoto et al. (1980) Cell 22:787-797), the herpes thymidine kinase promoter (Wagner et al. (1981) Proc. Natl. Acad. Sci. U.S.A. 78:1441-1445), the regulatory sequences of the metallothionein gene (Brinster et al. (1982) Nature 296:39-42), etc. Any type of plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product. See, e.g. Sarver et al. (1990) Science 247:1222-1225.

Ribozymes are enzymatic RNA molecules capable of catalyzing the specific cleavage of RNA. For a review, see Rossi (1994) Current Biology 4:469-471). The mechanism of ribozyme action involves sequence specific hybridization of the ribozyme molecule to complementary target RNA, followed by an endonucleolytic cleavage event. The composition of ribozyme molecules should include one or more sequences complementary to the target gene mRNA, and should include the well known catalytic sequence responsible for mRNA cleavage.

As in the antisense approach, the ribozymes can be composed of modified oligonucleotides (e.g. for improved stability, targeting, etc.) and should be delivered to cells which express the cystatin gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes unlike antisense molecules, are catalytic, a lower intracellular concentration may be required for efficiency.

Endogenous cystatin gene expression can also be reduced by inactivating or “knocking out” the targeted cystatin gene or its promoter using targeted homologous recombination. Smithies et al. (1985) Nature 317:230-234; Thomas and Capecchi, (1987) Cell 51:503-512; and Thompson et al. (1989) Cell 5:313-321. For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi (1987) and Thompson (1989), supra). For example, cystatin gene of livestock can be knocked out to produce animals that have a lower risk of transmission of tick infestation, animals where detection of tick infestation occurs immediately, or soon after tick infestation (at a time point earlier than in untreated animals), and animals in which ticks have a decreased ability to feed. However this approach can be adapted for use in humans provided the recombinant DNA constructs are directly administered or targeted to the required site in vivo using appropriate viral vectors.

The inactivation of cystatin should happen either in ticks (sialostatins are tick genes) or transformation of animals with a cystatin-neutralizing gene (e.g. RNAi or inhibitory compositions).

Anti-sense RNA and DNA, and ribozymes molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

With regard to the above, it is noted that reducing the in vivo activity of a particular sialostatin may relate to a reduction in the expression of a particular sialostatin or may relate to a reduction of the in vivo activity of a particular expressed sialostatin.

### Overexpression

For certain applications,.it may be desirable to overexpress a sialostatin L2 protein, for example in bacteria. Bacterial overexpression of sialostatin L has been described previously (29), and similar methods may be employed for overexpression of sialostatin L2. In certain preferred examples, removal of the leader sequence of sialostatin L2 is required prior to overexpression in bacteria.

### Dosage and Administration

The methods of the invention include immunogenic compositions. The immunogenic composition may further contain adjuvants, preservatives, chemical stabilizers, or other antigenic proteins. Typically, stabilizers, adjuvants, and preservatives are optimized to determine the best formulation for efficacy in the target human or animal. Suitable exemplary preservatives include chlorobutanol, potassium sorbate, sorbic acid, sulfur dioxide, propyl gallade, the parabens, ethyl vanillin, glycerin, phenol, and parachlorophenol.

One or more of the described immunogenic components may be admixed or adsorbed with a conventional adjuvant. The adjuvant is used to attract leukocytes or enhance an immune response. Such adjuvants include, but are not limited to, Ribi, mineral oil and water, aluminum hydroxide, Amphigen, Avridine, L121/squalene, D-lactide-polylactide/glycoside, pluraonic plyois, muramyl dipeptide, killed Bordetella and saponins, such as Quil A. In addition, a vaccine composition of the invention may further comprise other, non-B. burgdorferi antigens, or other vaccinal antigens originating from other species may also be included in these compositions.

Suitable amounts of the antigen can be determined by one of skill in the art based upon the level of immune response desired. In general, however, a protein-based immunogenic composition contains between 1 ng to 1000 mg antigen, and more preferably, 0.05 μg to 1 mg per L of antigen. Generally, a DNA-based immunogenic composition contains a peptide or protein antigen of the invention optionally under the control of regulatory sequences. Where the antigen-encoding DNA is carried in a vector, e.g. a viral vector, a dose may be in the range of 1×10⁻³ pfu to 1×10¹² pfu.

Other suitable does of the immunogenic composition of the invention can be readily determined by one of skill in the art. Generally, a suitable dose is between 0.1 to 5 mL of the immunogenic composition. The frequency of administration is to be determined by the practioner. For example, the immunogenic composition may be administered 1, 2, 3, 4 or more times, or on a seasonal basis (e.g. four times a year), and then once each tick season a booster would be administered. The immunogenic composition may be administered by any suitable route. However, parenteral administration, particularly intramuscular, and subcutaneous, is the preferred route. Also preferred is the oral route of administration. Routes of administration may be combined, if desired, or adjusted. Further, depending upon the human patient or the animal species being treated, i.e., its weight, age, and general health, the dosage can also be determined readily by one of skill in the art.

In general, the immunogenic compositions of the invention will be administered in the methods as claimed, for example in a method for the detection or for the prevention of a tick infestation in a subject, 1, 2, 3, 4 or more times a year. In general, the compositions will be administered for the first time, and then as many successive time to reach satisfactory immunity.

### Subjects

The present invention provides a wide variety of methods for using the therapeutic agents of the invention. These methods may be used with any type of animal. In one embodiment, the animal is a vertebrate. In another embodiment, the animal is a mammal. Specific examples of animals with which the methods, compositions and kits of the present invention may be used include, but are not limited to humans; livestock, such as chickens, turkeys, ostriches, ducks, geese, cattle, pigs, and horses; pets, such as cats, dogs, and horses; and animals that might be held in a zoo.

### Examples

### Example 1

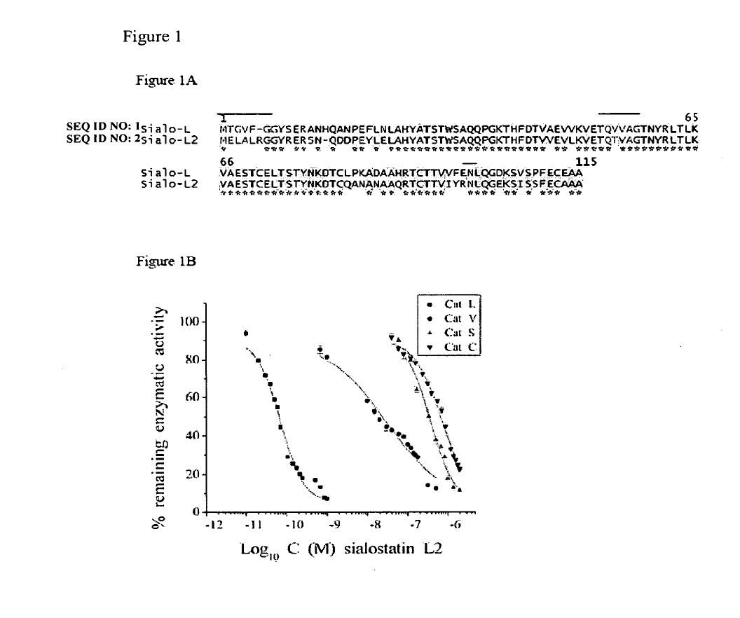

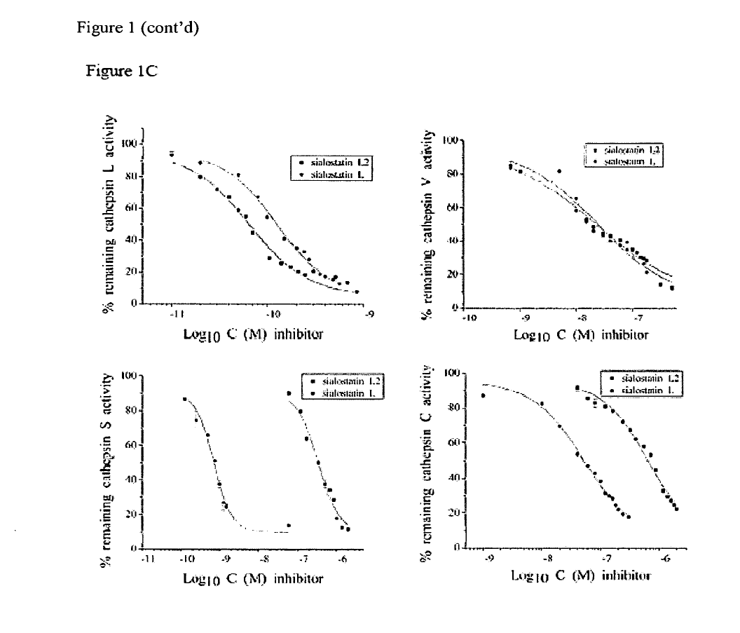

The two cystatin transcripts are encoded by two different genes. Several I. scapularis transcripts were revealed to be of salivary origin during a recent massive EST sequencing project (8) including a novel cystatin that shows 75% identity at the protein level to sialostatin L, a secreted cystatin previously characterized (9). When the secretion signal is removed from this polypeptide, multiple alignment with sialostatin L showed a clustering of aa substitutions in two regions of the protein; of a total of 27 aa substitutions throughout the 115 residue polypeptide, 12 were located in the first 22 amino terminal residues, while another 12 substitutions gather in the last 33 carboxy terminal aa of the protein, as shown in FIG. 1A. This result raised the possibility that the two proteins could be allelic products of the same gene. To test this hypothesis, a bioinformatic approach was undertaken. cDNA sequences of both transcripts were compared by BLAST analysis to the publicly available shotgun genomic sequences from the I. scapularis genome project. The resulting matches were assembled into contigs that were in turn compared by BLAST to both cystatin transcripts. The result showed that the two cystatins are encoded by two different genes (data not shown). The sialostatin L locus consists of three exons, while only two exons coding for parts of the amino terminus and carboxy terminus of the second cystatin could be revealed (data not shown). Possibly the third exon was not detected due to the limited DNA sequence available. In both genes, intronic sequences were partial but unique; their high numbers of repeating sequences made impossible their successful extension due to the very large number of matches with repetitive sequences from intronic regions found in the shotgun genomic sequences.

Members of the cystatin superfamily have been isolated from tissues of animals and plants and a variety of microbes. They can be subdivided into three groups (16); family 1 cystatins (also known as stefins) are cytoplasmic and lack disulfide bonds, while family 2 cystatins are secreted and bear two disulfide bonds. Members of both groups display low molecular weight (roughly 11-14 kDa) in contrast to the family 3 members (also known as kininogens) that are much larger molecules made of multiple cystatin modules. Structural studies of various cystatins show that they display a wedge-shaped interface that binds to the active site of their target proteases (17). This interface consists of three typical segments (18) (shown in F(G. 1A): the N-terminal domain located around a conserved G (PI segment); a hairpin loop located around the conserved sequence QXVXG (PII segment); and a second hairpin loop located around a conserved PW dipeptide (PIII segment).

### Example 2

The polypeptide products of the two genes differ in their target specificity and display different antigenicity. Next, expression and purification of the protein encoded by the novel transcript was carried out, which was subsequently used in inhibition assays of various commercially available purified proteases. FIG. 1B shows that four cysteine proteases of seven tested were affected by the presence of the protein in the assay, including cathepsins L, V, S, and C. Inhibition was not observed for cysteine proteases cathepsin X/Z/P, B, or H (Table 1, below), aspartic proteases cathepsin D and legumain, or serine proteases cathepsin G and elastase (data not shown). Table 1 shows Sialostatin L2 affinity changes for proteolytic enzymes when compared with sialostatin L. In Table 1, a repertoire of cysteine proteases were tested for inhibition by sialostatins L and L2 and the concentration of inhibitor at which 50% inhibition of the activity of the targeted proteolytic enzymes is achieved. (IC50)±standard error are presented. Enzyme concentration used in the assays is also given for all their targets. NI, no inhibition, i.e., inhibition of the enzyme was not observed in the presence of 10 μM inhibitor.

TABLE 1

| | Enzyme | | |
|---|---|---|---|
| Enzyme | Concentration | Sialostatin L2 IC₅₀ | Sialostatin L IC₅₀ |
| Cathepsin L | 20 pM | 70.9 ± 3.4 pM | 125.8 ± 3.7 pM |
| Cathepsin V | 1 nM | 28.8 ± 2.8 nM | 24.1 ± 2.7 nM |
| Cathepsin S | 60 pM | 378.1 ± 23 nM | 0.7 ± 0.01 nM |
| Cathepsin C | 10 nM | 740.4 ± 22.3 nM | 52.2 ± 2.3 nM |
| Cathepsin | 16 nM | N.I. | 937 ± 14 nM |
| X/Z/P | | | |
| Cathepsin B | | N.I. | N.I. |
| Cathepsin H | | N.I. | N.I. |

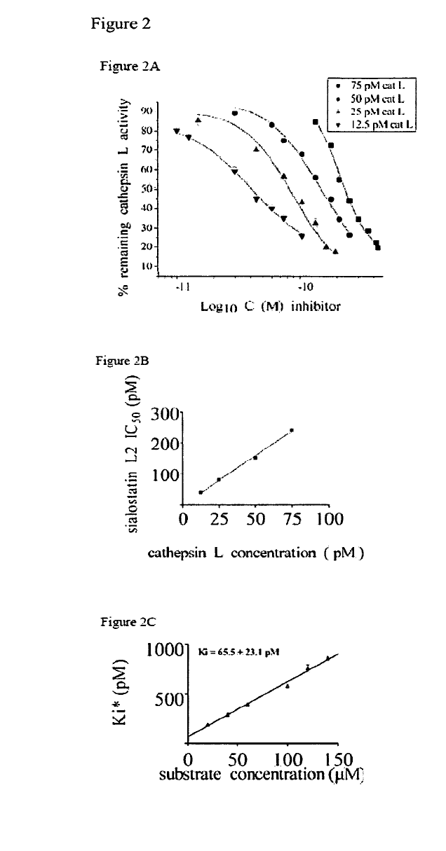

Next, this novel cystatin was compared with sialostatin L for efficiency in inhibiting their overlapping target enzymes. The results are shown in FIG. 1C and summarized in Table 1, above. Briefly, the two inhibitors are equally potent for inhibition of cathepsins L and V (FIG. 1C, upper panel) but displayed major differences in inhibition of cathepsins S and C (FIG. 1C, lower panels). To further evaluate those findings, it was tested whether this novel cystatin is a tight inhibitor for cathepsin L, as is the case for sialostatin L (9). Indeed, when decreasing amounts of cathepsin L were used in the assays, less cystatin was necessary to achieve the same percentage of enzymatic inhibition (FIG. 2A), which is a typical characteristic of tight inhibition. The decrease in the concentration of the inhibitor at which 50% enzymatic inhibition (IC50) is achieved was actually analogous to the reduction of the amount of enzyme used in the assay (FIG. 2B). Because conventional Michaelis-Menten kinetics do not hold true for tight binding inhibition, we applied Morrison's equation (12) to obtain apparent dissociation constants (Ki*) in the presence of varying substrate concentrations. FIG. 2C shows the linear regression line (r2=0.9918) when Ki* for several substrate concentrations was plotted against the substrate concentration, indicating a y intercept of 65.5±23.1 pM that is the inhibition constant (Ki) of this novel cystatin for cathepsin L. The sialostatin L Ki for the same enzyme is 95.3±7.3 pM (9), demonstrating a similar affinity of the two inhibitors for cathepsin L. To emphasize this similarity, the name sialostatin L2 was given to this second salivary cystatin.

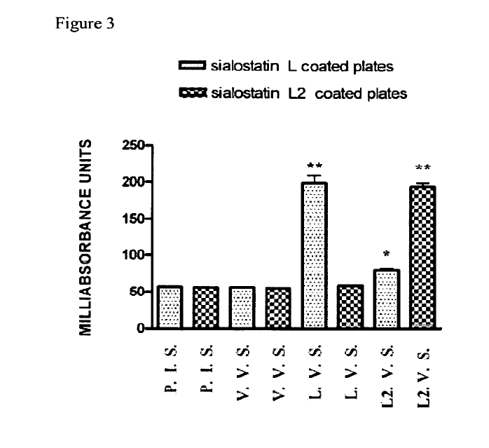

Having in hand both pure and active cystatins, their antigenicity, i.e., their capability to induce production of specific polyclonal sera in a vertebrate host, in this case female Swiss Webster mice, was examined next. Sialostatin L or L2 was administered (20 μg) in each mouse five times at 2-wk intervals; 2 wks post the last vaccination, their sera were tested by enzymelinked immunosorbent assays (ELISA) for recognition of vaccination antigen (sialostatin L or L2) and potential for crossreaction with the second cystatin (sialostatin L2 or L, respectively). The results are shown in FIG. 3. While both proteins were immunogenic, only sera from mice vaccinated with sialostatin L2 crossreacted with sialostatin L. In a step further the mean antibody titer in the sera was estimated for the mice in both experimental groups, using standard methods (11); for the sialostatin L vaccinated mice the mean antibody titer was 4100±400 for sialostatin L and 200±35 for sialostatin L2, while the mean antibody titer in the sera of the sialostatin L2 vaccinated mice was 4000±450 for sialostatin L2 and 1070±136 for sialostatin L.

In summary, the 27 different aminoacids between the two cystatin molecules apparently results in changes in their interaction interface with some of the targeted enzymes (and therefore their binding affinity). These primary structure changes and more interestingly, the observed different affinity of the two inhibitors for cathepsin S—a critical enzyme for antigen processing and presentation—can account for the observed differences in their recognition from the vertebrate immune system as well.

Referring again to the structural studies of various cystatins that show that secreted cystatins from ticks are divergent in their aa sequence from the other family 2 members from animals and lower eukaryotes (9), it is shown here that both I. scapularis salivary cystatins lack the two PW residues in the PIII segment that are instead substituted with a conserved NL dipeptide. Single amino acid (aa) substitutions in the PW dipeptide have been shown to reduce cystatin affinity for cathepsins B and H (19). It is possible that sialostatins L and L2 recruited those two aa substitutions for I. scapularis to get rid of a potentially undesirable or unnecessary inhibitory activity of their salivary cystatins against vertebrate cathesins B and H, which could diverge these salivary proteins for their target selectivity.

### Example 3

Sialostatin L2 transcription increases as feeding to the host progresses. To shed light on transcriptional control of the two genes during I. scapularis feeding on the vertebrate host, real-time quantitative RT-PCR using RNA isolated from unfed or partially fed adult female tick salivary glands or midgets was employed. Expression levels were first normalized using the constitutively expressed actin transcript as a standard (14). Similar accumulation of sialostatin L transcripts was revealed in unfed salivary glands and midguts, 80 and 20 times higher when compared with sialostatin L2 expression levels in the corresponding tissues. Furthermore, the difference in transcript abundance for the two tick cystatins, both in the midgut and in the salivary glands, as feeding continues and when compared with the corresponding transcript abundance in tissues from unfed ticks was estimated and it is presented in Table 2, shown below. Table 2 shows Transcriptional regulation of sialostatins L and L2 in the midgut and salivary glands during the onset of tick blood feeding. The table shows the difference in accumulation of transcripts for the two tick cystatins, both in the midgut and in the salivary glands, as feeding continues and when compared with the corresponding transcript abundance in tissues from unfed ticks. Similar levels of sialostatin L transcripts were revealed in unfed salivary glands and midguts, 80 and 20 times higher when compared with those of sialostatin L2 in the corresponding tissues.

TABLE 2

| | Sialostatin L2 | | Sialostatin L | |
|---|---|---|---|---|
| | (Fold Difference) | | (Fold Difference) | |
| Feeding Period | Midguts | Salivary Glands | Midguts | Salivary Glands |
| Unfed | 1 | 1 | 1 | 1 |
| 24 Hours | 1.3 | 29 | 0.3 | 0.3 |
| 48 Hours | 1.2 | 273 | 0.1 | 0.1 |
| 72 Hours | 0.3 | 232 | 0.1 | 0.2 |
| 96 Hours | 1.3 | 940 | 0.03 | 0.1 |

Briefly, as feeding starts, sialostatin L transcript levels decrease in both the midgut and salivary glands. On the other hand, sialostatin L2 transcripts slightly fluctuate in the midgut but drastically accumulate in salivary glands. This bioinformatics approach uncovered that the 600 bp of the 5′UTR of the two genes do not show any similarity when compared with BLASTN (data not shown), indicating that the differences in the transcription regulation of the two genes can be partially or fully attributed to their different 5′UTR nucleotide sequences.

### Example 4

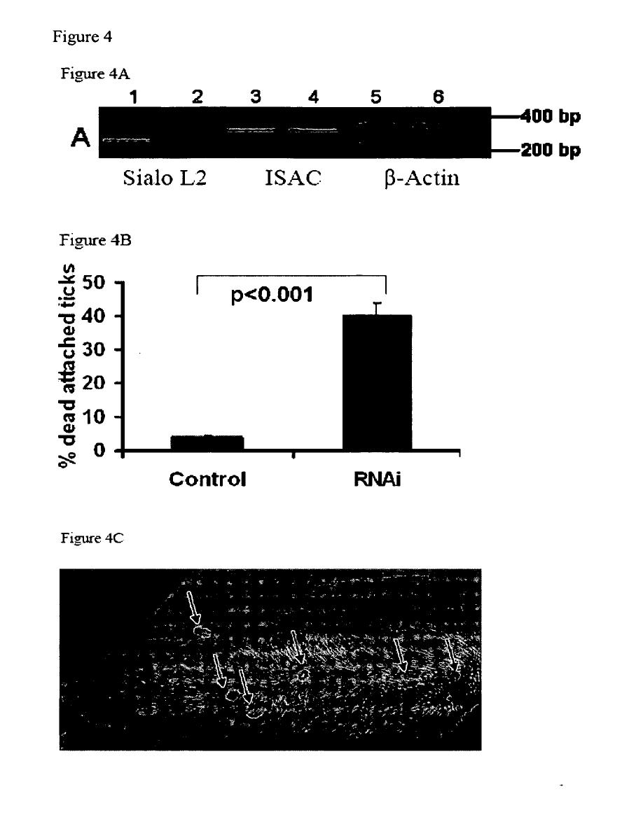

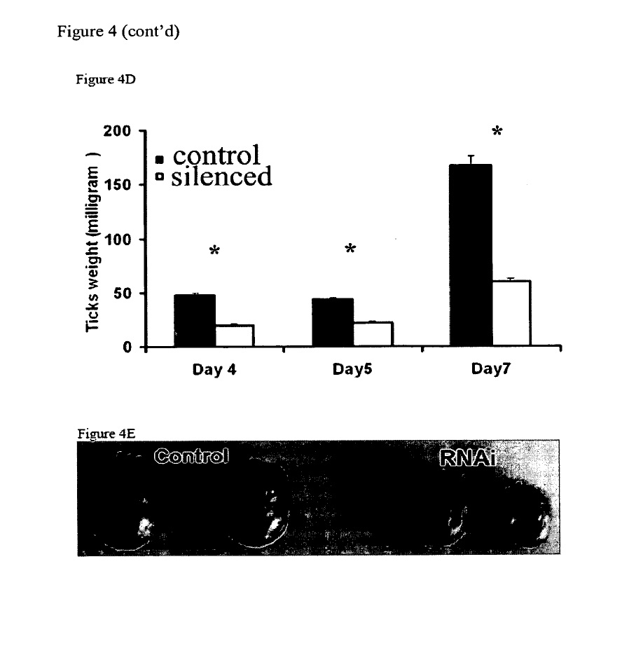

Sialostatin L2 is essential for tick blood feeding success. Given this transcriptional induction of sialostatin L2 in tick salivary glands as feeding progresses, next RNAi was used to silence the gene. Adult unfed female ticks were injected with sialostatin L2 dsRNA and subsequently allowed to recover from the injections and feed on rabbits as described in Methods, below. Groups of 12 ticks each were pulled from the rabbit after 4 days of feeding and their salivary glands were dissected and subsequently checked for gene silencing efficiency by RT-PCR. As shown in FIG. 4A, ticks injected with sialostatin L2 dsRNA showed an approximately 80% decrease in sialostatin L2 transcript levels when compared with water-injected controls. Moreover, sialostatin L was completely silenced (data not shown), while levels of β-actin and Isac (negative controls) remained unchanged in both experimental and control groups. When attached to a rabbit in vivo, ˜40% of the silenced ticks were unable to feed and subsequently died (FIG. 4B), while in most cases apparent inflammatory and swollen skin was revealed in the feeding sites of dead ticks (FIG. 4C). For the remaining ˜60% of RNAi ticks that fed on the host to repletion, their average weight approximated 60 mg, much lower than the control average weight of 170 mg (FIGS. 4D and 4E). Additionally, they showed ˜70% egg-laying inhibition and became ‘stone hard’ after detachment from the host (data not shown).

### Example 5

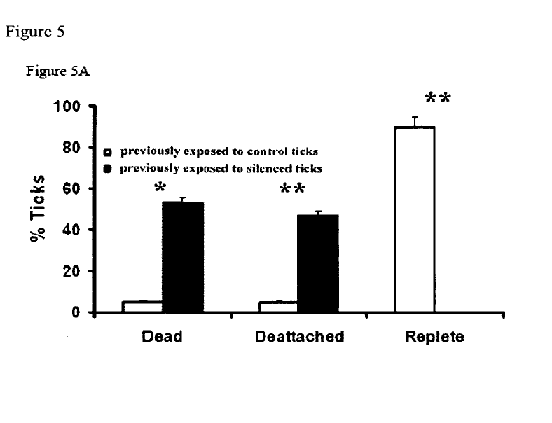

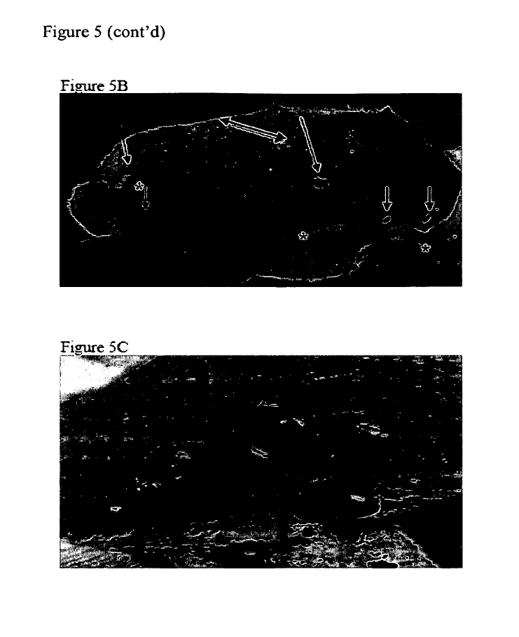

The phenotype of silenced ticks can be attributed to enhanced immune reaction from the host. Rabbits exposed multiple times to ticks eventually develop a strong anti-tick immunity (15). It was hypothesized that the signs of inflammation in feeding sites of dead ticks treated with cystatin dsRNA could indicate an accelerated immune response to tick salivary proteins because sialostatins are absent or decreased. Therefore, rabbits exposed to control and silenced ticks were kept and exposed to wild type (normal) adult female ticks 2 wk after the first infestation. As shown in FIG. 5A, when ticks were attached to rabbits previously exposed to RNAi-treated ticks, they fed poorly and were unable to engorge, while a severe skin reaction could be seen at the tick attachment site (FIG. 5B). In contrast, when adult female ticks were attached to rabbits previously exposed to water injected control ticks, they managed to feed and engorge (FIGS. 5A and 5C), although less efficiently (data not shown) than when attached on na\ddot{v}e rabbits (never exposed to ticks), in agreement with a previous report (15).

Identity of the two cystatins at the amino acid level suggests that the corresponding genes resulted from a relatively recent duplication event. The question arises why such an event was fixed in the genome. Both inhibitors target the same proteases, namely cathepsins L, V, S, and C, but on 7 the other hand, they differ in their affinity for cathepsins S and C. Additionally, antisera produced against the two proteins were not completely crossreactive. Furthermore, there were differences in their transcriptional regulation; sialostatin L2 transcripts rapidly and constantly accumulate as feeding progresses. Given this induction of sialostatin L2, there is possibly enhancement of the inhibitory activity of saliva against cathepsins L, V, C, and S as feeding to the host continues, assuming that transcript accumulation will result in a corresponding increase of sialostatin L2 secretion from the salivary glands.

Ticks can be considered clever pharmacologists (22), because adaptation to their natural vertebrate hosts has sculptured their saliva composition in such a way that the amount of each salivary constituent is sufficient to counteract any host action that would lead to tick rejection. Cathepsins V, L, and S are efficient elastinolytic endopeptidases identified as secreted by macrophages during the onset of inflammation (23) and as major contributors to tissue damage under chronic inflammatory conditions (24). Elastic fibers are important extracellular matrix components conferring elasticity to tissues such as blood vessels and skin. In the absence of salivary cystatins, proteolytic degradation of elastic fibers resulting from the release of cathepsins in the initial steps of tick infestation would destroy tissue elasticity and lead to high risk for maintaining the tick feeding cavity. This is the phenotype of the RNAi ticks: immediate rejection or failure to successfully accomplish a blood meal. This phenotype can be further explained from extensive work on the role of cathepsins L and S in antigen presentation/immunity (25,26). Absence of immunosuppressive action of cystatins during the first infestation (the genes were knocked down by RNAi) led to a much stronger primary immune response from the vertebrate host as shown by the increase in the number of dead ticks and the signs of inflammation in their attachment sites. Subsequent boost of the same animal with a second tick infestation had detrimental consequences for tick feeding, as shown by almost immediate tick rejection and stronger inflammatory responses in the sites of infestation.

Previous work has shown the importance of anticoagulation in I. scapularis feeding success using an RNA interference approach (27). In the results presented herein, in addition to confirming the value of the technique in gene function analysis in this nonmodel organism, bioinformatics/genomics, biochemistry, and molecular biology are combined to shed light on the mechanism of action of another key mediator in the tick strategy to access the bloodstream for a long time without triggering host reactions; saliva cystatins target a limited number of vertebrate cysteine proteases that possess an important role in vertebrate immunity.

The results presented herein presents an analysis of cystatins vis a vis their target specificity, making them useful tools in the study of their target enzymes in various biologic phenomena. Moreover, extensive work involving transgenic mice that lack the corresponding gene(s) has shown the implication of cathepsins L and S under various pathologic conditions including atherosclerosis and cancer (28,29). Because of its stringent and unique specificity, the sialostatins, in particular sialostatin L2, can be useful for studying the role of certain papain-like proteases in various biologic phenomena. In addition it can provide a starting point for potent pharmaceutical interventions that target the key role of those enzymes in human diseases. Besides their limited number of targets, the results herein reveal the crucial mediation of I. scapularis cystatin salivary constituents in bloodmeal uptake through control of their targets' proteolytic activity. Taking into account their role in the success of parasitism, they should be considered in the development of antiparasitic vaccines; they may be additional candidate ingredients in the cocktail of antigens that will potentially lead to achievement of this difficult goal.

### Example 6

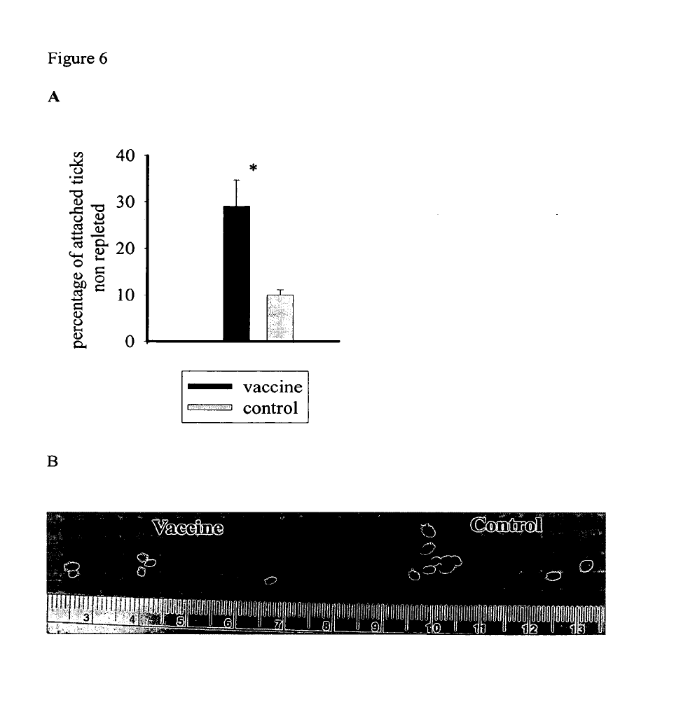

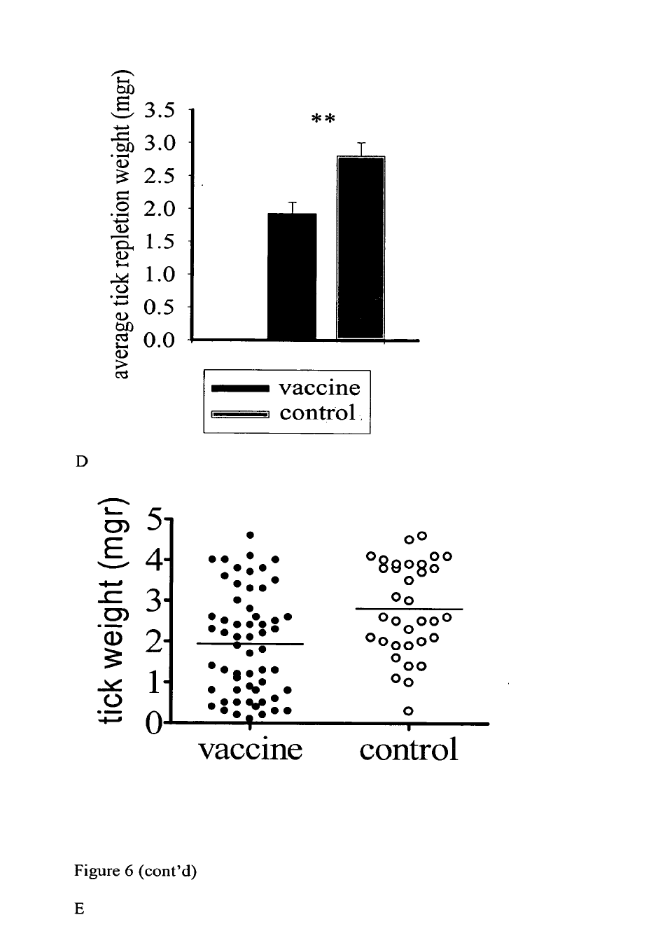

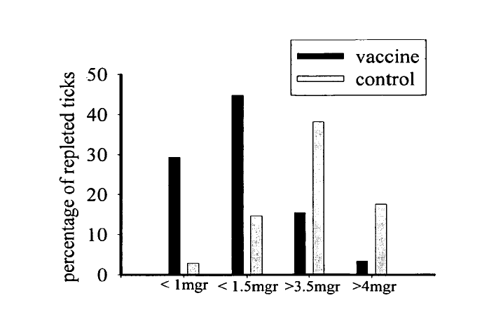

Impaired feeding of Ixodes scapularis nymphs in Guinea Pigs vaccinated with Sialostatin L2. Nymphs are the developmental stage of Ixodes scapularis that are highly associated with disease transmission, mainly due to their small size that makes it difficult to notice their attachment to a subject. Here, Guinea Pigs (GPs) were vaccinated intradermally with 100 micrograms of sialostatin L2 4 times, in 2 week intervals. I. scapularis nymphs were attached to the shaved heads of GPs 15 days post the last vaccination. As shown in FIG. 6A, 3 times more ticks failed to attach or blood feed in the sialostatin L2 vaccinated group (29% of the total nymphs administered to the GPs) when compared to the control group (10% of the total nymphs failed to feed respectively). The rest of the ticks that managed finally to feed received more blood when feeding to control GPs. FIG. 6B shows a characteristic photo of the difference in the size of the first engorged ticks found in the bottom of the cage 4 days post nymphal attachment to the GPs. This difference continued throughout the feeding period of nymphs, resulting an average tick repletion weight of 1.9 mg for the nymphs fed on sialostatin L2 vaccinated GPs, compared to the corresponding 2.8 mg for the nymphs fed on the control group (FIG. 6C). Of note, the difference would be greater if the calculation took into account the ticks that were immediately rejected. FIGS. 6D and 6E show the distribution of tick weight in the control and sialostatin L2 vaccinated group. FIG. 6E demonstrates that only 3.4% of the ticks fed on the sialostatin L2 vaccinated GPs exceeded 4 mgs (compared to 17.6% in control) and 15.5% exceeded 3.5mg (compared to 38.2% in control). On the other hand 29.3% weighed lesser than lmg (compared to 2.9% in control) and 44.8% weighed lesser than 1.5mgr (compared to 14.7% in control).

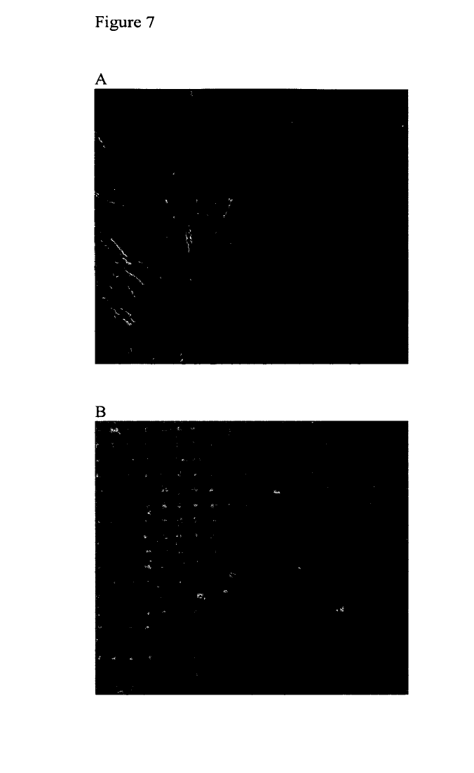

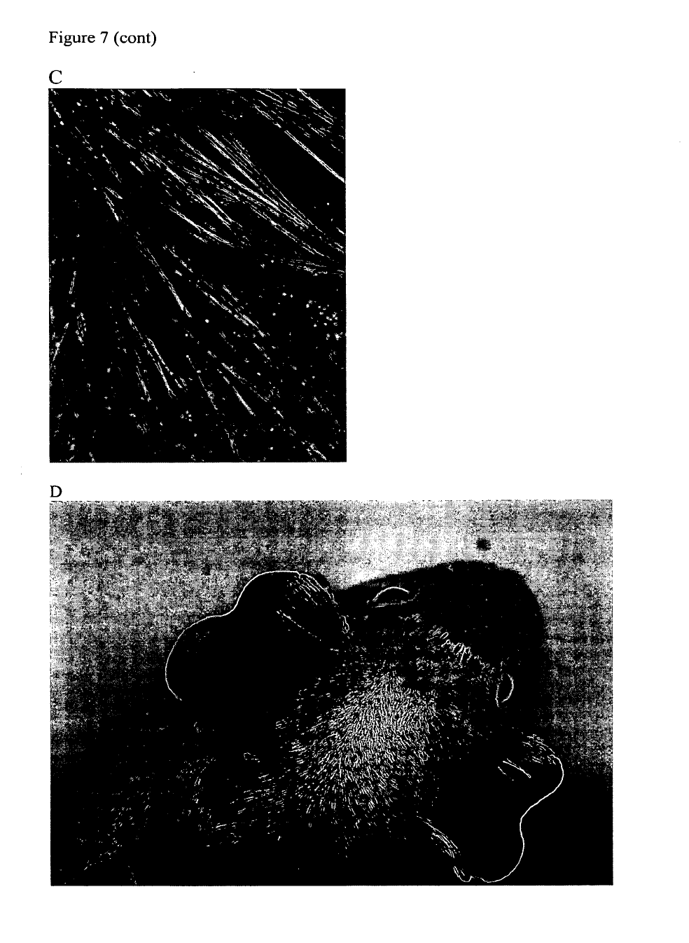

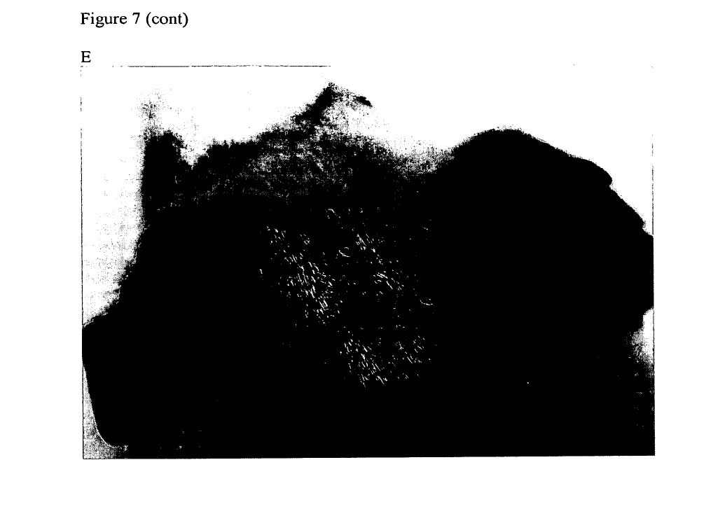

Moreover, signs of inflammation developed in the sites of nymphal infestation in the sialostatin L2 vaccinated GPs 24-96 hours post tick attachment, as shown in FIGS. 7A-7C. Red and swollen skin developed in the sialostatin L2 vaccinated group accompanied by bleeding and blisters. FIG. 7D-7E shows characteristic photos from the heads of sialostatin L2 vaccinated and control GPs 96 hours post nymphal attachment.

The data presented herein demonstrates that presensitizing vertebrate immunity against a tick salivary immunomodulatory, sialostatin L2, leads to impaired blood feeding by the Lyme vector I. scapularis.

Taken together, this series of experiments shows that the RNAi results recapitulate the results obtained from treatment with a sialostatin L2 immunogenic composition. Sialostatin L2 possesses a critical and rather conserved role during nymphal feeding of Ixodes scapularis in Guinea Pigs. The reduction of the blood taken from the nymphs can have a detrimental effect to their developmental potential into adults, while the acute inflammation in the sites of their attachment would put the pathogens they transmit in a rather unfriendly environment, or an environment that is not conducive to transmission of disease.

Considering that it is almost impossible for the Guinea Pigs to remove the ticks from their heads, translating the results to a human subject would lead to a different fate for the ticks. The edema, inflammation and pain seen in the GP model may be an effective means for detection in the human host.

### Example 7

Impaired blood feeding of Ixodes scapularis on hosts immunized against one of its salivary immunomodulators and multicomponent imunogenic compositions. As previously described, Ixodes scapularis nymphs are the key vector stage implicated in Lyme transmission in disease endemic regions of the US, mainly due to their small size that makes timely detection difficult. A variety of strategies such as the adoption of acaricide based programs or the use of repellents have been proposed for control of tick populations, but the high cost of implementation and potential for mammalian toxicity and environmental damage are among the reasons that fewer than 25% of residents in these endemic areas report spraying of their properties to control ticks. On an ecologic level, the absence of natural tick predators or enemies has led to introduction of vegetation management and application of desiccants or soap to control tick populations, in addition to personal protection measures such as wearing appropriate clothing. Additional attention has been drawn lately to protection of wildlife and companion animals (such as rodents, deer, and dogs) because they can both provide a nutritious meal essential for tick survival and serve as pathogen reservoirs. Despite most of the above measures, the control of tick borne diseases has not yet been achieved.

Another alternative or complementary constituent of such an integrated approach against ticks and/or the pathogens they transmit is development of anti tick vaccines. This idea is mainly supported from the phenomenon that certain vertebrate hosts develop tick hypersensitivity upon repeated exposure to ticks, preventing ticks from taking a blood meal. This anti tick immunity can, upon tick re exposure, prevent Borrelia transmission, as well (Nazario, S., S. Das, A. M. de Silva, K. Deponte, N. Marcantonio, J. F. Anderson, D. Fish, E. Fikrig, and F. S. Kantor. 1998. Prevention of Borrelia burgdorferi transmission in guinea pigs by tick immunity. Am J Trop Med Hyg 58:780-785.).

A number of salivary compounds are present in tick saliva, and have been, and continue to be characterized. Further, some of these compounds exert their action on the host in low nanomolar to picomolar concentration. Sialostatin L2 also functions at these low nanomolar to picomolar concentrations.

This is not surprising, as intense work on the transcriptome and proteome of I. scapularis glands (6) has revealed numerous components of its saliva and, more importantly, their potential pharmacologic action on physiological phenomenon such as host coagulation, fibrinolysis, immunity, and angiogenesis. This action not only facilitates tick attachment to the vertebrate host and recruitment of a good quality meal, but also creates a tick/vertebrate host interface advantageous for pathogen transmission. Such a pathogen transmission facilitation has already been demonstrated for Salp15, a tick salivary immunomodulator exploited during Lyme disease transmission (Ramamoorthi, N., S. Narasimhan, U. Pal, F. Bao, X. F. Yang, D. Fish, J. Anguita, M. V. Norgard, F. S. Kantor, J. F. Anderson, R. A. Koski, and E. Fikrig. 2005. The Lyme disease agent exploits a tick protein to infect the mammalian host. Nature 436:573-577). Therefore, vertebrate host immunity that blocks pharmacologic action of tick salivary constituents has the potential to affect tick feeding ability and transmission of tick borne pathogens.

Accordingly, the low amount of protein necessary for the inhibition of its target enzymes, led to further experiments to determine whether naturally immunized guinea pigs (exposed to ticks for four times) recognize sialostatin L2 tick secretion. It was found that humoral recognition could not be detected and therefore the term ‘silent’ antigen was introduced for sialostatin L2 to describe the fact that although the protein is found in the tick-host interface, it can not be recognized by humoral vertebrate immunity upon repeated tick infestations. One possibility for this is due to its function, or to the amount of its secretion.

Presensitizing

The experiments described herein demonstrate that presensitizing vertebrate immunity against a tick salivary immunomodulatory, for example sialostatin L2, leads to impaired blood feeding by the Lyme vector I. scapularis. The experiments show proof of principles for a vaccine to be developed, aiming to neutralize the detrimental (for the vertebrate host) action of this salivary gland antigen. The experiments described herein demonstrate an integrated approach for controlling the diseases that are transmitted by this vector in endemic areas of the US.

In the experiments, administering supraphysiological amounts of immunological composition leads to neutralized action of Sialostatin L2 and to impaired blood feeding by I. scapularis nymphs. The experiments shown reveal an essential immunosuppressive action of sialostatin L2 upon nymphal infestation that can be blocked by vertebrate humoral immunity.

Guinea pigs, but not their natural hosts, develop immune mediated tick hypersensitivity after repeated exposure to I. scapularis with a mechanism similar to humans (75). Although sialostatin L2 is a secreted salivary protein and its transcripts were detected in nymphal salivary glands (76), it was not possible to detect any sialostatin L2 recognition by ELISA assays or western blots using sera from guinea pigs exposed to I. scapularis nymphs four times in 2 wks intervals (data not shown). Further experiments showed that recognition of higher molecular weight salivary gland antigens could be detected by both ELISA and western blots using the same sera as a primary Ab and salivary gland homogenates as antigens. It is possible that the low amount of protein necessary to exert its action (77) upon tick infestation accounts for this unexpected result. Thus, the immunogenicity of the protein when injecting supraphysiological amounts in guinea pigs was next tested.

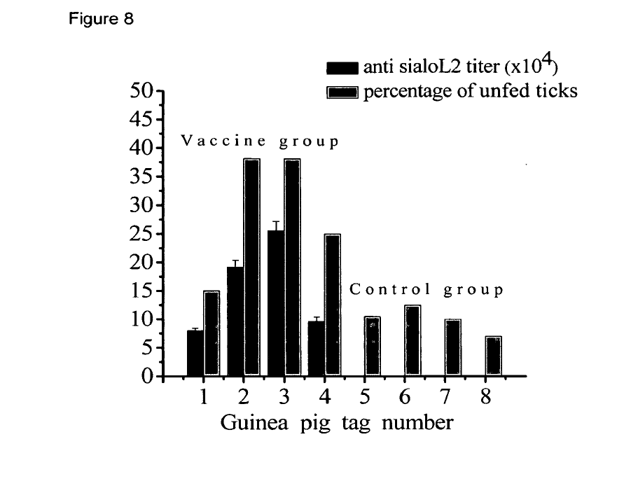

Four animals were vaccinated intradermally with sialostatin L2 protein in a considerably higher amount (100 μg) than nymphal salivary glands can secrete upon tick infestation. Two wks post vaccination, sera samples were prepared from the animals and tested by ELISA for sialostatin L2 recognition. An average anti-sialostatin L2 titer of 156±41.5×103 (n=4) was achieved, ranging between 8×104 to 25.6×104 in the sialostatin L2 vaccinated animals (FIG. 8).

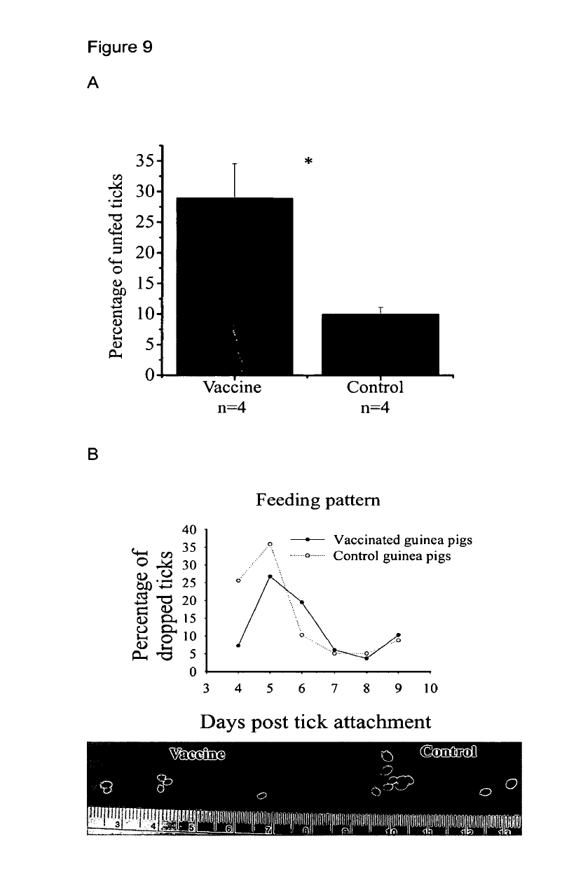

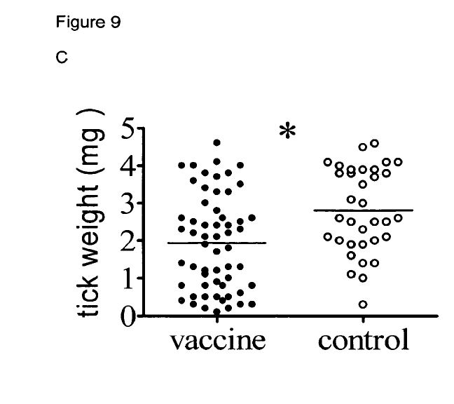

Having presensitized the animals for a tick salivary gland antigen, the animals were subsequently exposed to I. scapularis nymphs by administering 20 ticks in each animal (see Material and Methods). All of the nymphs that were not attached to the animals within the first 3 hours of placement were removed using forceps. During the first three days of tick exposure all nymphs found on the bottom of the animal cage that had a body weight similar to that before attachment to the guinea pigs were collected and counted and were considered to be “unfed” as a consequence of early rejection (within the first 72 h of exposure). FIG. 9A shows that increased early rejection was observed for the ticks attached to the sialostatin L2 vaccinated group. The average early rejection rate in the vaccine group (n=4) was 29±5.6%, three times higher than that in the control group (n=4) (10±1.1%; P=0.016). Higher early rejection percentage was observed for the ticks attached to animals displaying higher anti-sialostatin L2 titer (See FIG. 8).

The remainder of the ticks—those that were not rejected during the first 72 h—were able to receive blood and consequently increased their body weight. Approximately 15% of the ticks that fed on the vaccinated animals (and not those that fed on the control animals), triggered apparent signs of inflammation in their feeding sites, i.e., increased redness and edema formation, 72 h post attachment (FIG. 7C). Although this host reaction affected the feeding (and engorgement) of some ticks (FIG. 7C, upper tick), other ticks appeared unaffected by this local reaction (FIG. 7C, lower tick). This sign of apparent increased vertebrate host response to nymphal infestation was further supported by the delayed drop off of ticks feeding on the vaccinated group (P=0.03, χ2 for days 4-6) (FIG. 9B, graph). Most of the engorged ticks (data not shown) dropped between days 4 and 5 in the control group, but between days 5 and 6 in the vaccine group (FIG. 9B, graph). The delay in blood feeding was even more obvious when comparing the body size and weight of ticks that dropped off on day 4 (FIG. 9B, photo). While ticks from the control group were almost fully engorged, those recovered from the vaccine group appeared (and weighed) partially engorged. Moreover, FIG. 9C reveals that by the end of the nymphal feed, there was a statistically significant reduction (P=0.009) in mean weight of ticks feeding on the vaccine group compared with those feeding on the control group (1.9±0.2 mg and 2.8±0.2 mg, respectively).

An analysis of tick weight distribution is shown in Table 3, below.

TABLE 3

| | Percentage of total | Percentage of total |
|---|---|---|
| | nymphs | nymphs recovered |
| Nymphal weight | recovered from | from non vaccinated |
| group (mg) | vaccinated animals | animals |
| <1.5 | 44.8 | 14.7 |
| <1 | 29.3 | 2.9 |
| >3.5 | 15.5 | 38.2 |
| >4 | 3.4 | 17.6 |

Table 3 shows that a larger proportion of the nymphs that received a blood meal from the vaccinated guinea pigs (compared to the control) displayed low body weight, while a smaller proportion of them (compared to the control) received a good quality meal. 2.5 to 10 times more nymphs weighed less than 1.5 or 1 mg when attached to the vaccine group and more than 3.5 or 4 mg when attached to the control group.

Taken together, the data show that impaired I. scapularis nymphal feeding was observed upon attachment to sialostatin L2 vaccinated animals, which was the combined result of an early rejection of the nymphs and reduction in the ability of remaining nymphs to receive blood.

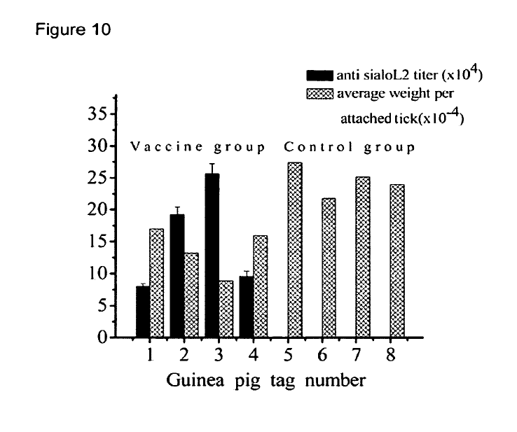

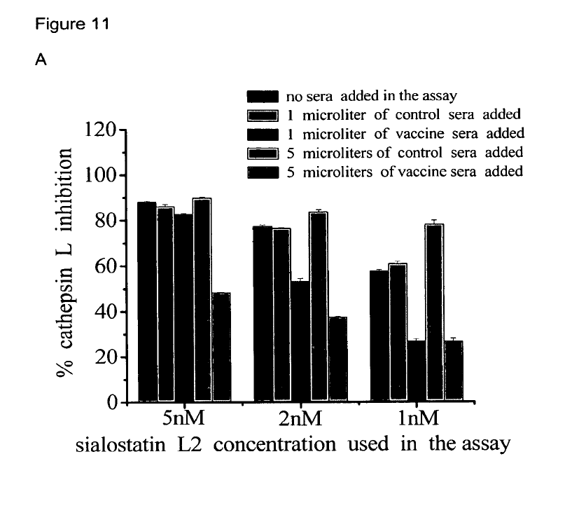

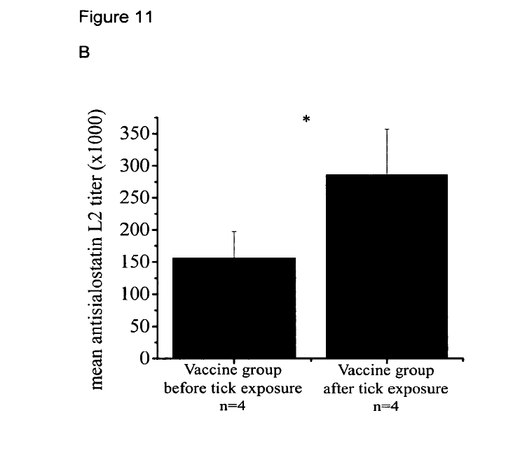

FIG. 10 incorporates both mechanisms contributing to the observed feeding impairment; the graph represents the average weight per nymph initially attached to each animal (taking into consideration calculations of the weight of the rejected nymphs in both groups). The graph shows that that there is a greater effect on ticks attached to guinea pigs with higher anti-sialostatin L2 titer. This observation led to the further investigation in vitro whether this Ab titer can also neutralize sialostatin L2 action, i.e., inhibition of cathepsins (77). Total IgGs were purified from animal #3 (sialostatin L2 vaccinated) and animal #5 (control) sera (see FIGS. 8, 10), prior to their exposure to ticks. As a result of the IgG purification procedure there was a 5-fold reduction in the titer of animal #3, which was estimated as 4.8×10⁴ ±0.6×10⁴. Subsequently, 5, 2, and 1 nM of sialostatin L2 were incubated in the presence or absence of 1 or 5 μl of purified IgG from animals #3 and #5 in 50 μl of cathepsin L assay buffer for 10 min (9). Subsequently, purified cathepsin L was added to the mix and incubated for another 10 min before addition of a fluorogenic substrate for estimation of cathepsin L enzymatic activity in triplicates (9). A statistically significant inhibition of sialostatin L2 activity on cathepsin L was observed upon addition of anti-sialostatin L2 IgG but not control IgG (FIG. 11A). The only exception was when incubating 1 μl of anti-sialostatin L2 IgGs with 5 nM of the protein that a statistically not significant reduction in protein activity was observed (FIG. 11A). Although the inhibitory activity of sialostatin L2 was not completely neutralized in our assays, this could be due to the fact that the pH of cathepsin L assay buffer is 5.5, which is not optimal for binding of Abs to their respective antigens.

Given the potential of the sera obtained from vaccinated animals to neutralize tick sialostatin L2 action, it was next tested whether this neutralizing recognition of the tick inhibitor by the vertebrate immune system took place upon guinea pig infestation with I. scapularis nymphs. Sera were prepared 2 wks after removal of the last tick from the guinea pigs, and their sialostatin L2 titers prior to and after tick exposure were compared in the same ELISA plate. As shown in FIG. 11B, a statistically significant ˜2 fold boost in the titer of the animals was revealed (from mean anti-sialostatin L2 titer of 156±41.5×103 before exposure to ticks to 286±71×103 after tick exposure, paired t-test P value=0.04). Moreover a statistically insignificant fluctuation of the anti-sialostatin L2 titer was observed in similarly vaccinated animals that were not exposed to ticks (control group, data not shown) collectively suggesting that sialostatin L2 secretion was recognized by the vertebrate immune system upon tick infestation on the vaccinated guinea pigs.

The increase in the number of cases of tick-transmitted diseases in humans has enhanced research effort towards tick host pathogen interaction studies and more specifically to molecular dissection of tick salivary secretion, which has already been shown to significantly facilitate transmission of pathogens. The continuous discovery of tick salivary constituents and their subsequent biochemical characterization highlights their potential pharmacological action on the vertebrate host in nanomolar or picomolar concentration as it is the case for sialostatin L2 (77). Therefore it was next determined whether this salivary effector is recognized by vertebrate humoral immunity upon repeated exposure to ticks; the lack of recognition led to the introduction of the term ‘silent’ antigens to cover all tick secreted salivary antigens that are not recognized by vertebrate immunity upon repeated exposure to ticks. The results shown herein demonstrate that pursuing similarly ‘silent’ antigens could reveal novel anti-tick vaccine antigens that could complement the four tick salivary antigens whose vaccines were shown to partially protect from Ixodes ricinus or I. scapularis infestation or/and the pathogens they transmit (78-81).

Over their evolution, adaptation of ticks, and blood feeding arthropods in general, to the vertebrate host has sculptured the composition and amount of their saliva constituents through a the process of selection at the population level. One conclusion may be that the survivors are thus those that ‘know well’ what they will face upon infestation of the vertebrate host. Described herein is a constituent of the salivary armamentarium of I. scapularis that goes undetected by the ‘radars of immunity’ in these sensitized animals. The discovery of tick hypersensitivity on certain vertebrate animals upon re exposure to ticks turned research efforts toward the tick antigens that dictate this hypersensitivity. Described herein are constituents of the salivary armamentarium of I. scapularis that is undetected in these naturally sensitized animals. The results presented herein indicate that it may be possible to administer to a subject a higher amounts of protein than those usually secreted by ticks upon infestation, and thus prevent tick infestation.

The idea for development of an anti tick vaccine was first successfully applied to protection of animal health and production by targeting the feeding ability of Boophilus spp. that infests cattle (Willadsen, P. 2006. Vaccination against ectoparasites. Parasitology 133 Suppl:S9-S25). The success of this approach and the increasing cases of tick transmitted diseases to humans brought enhanced research effort to tick host pathogen interaction studies and more specifically to molecular dissection of tick salivary secretion, which has already been shown to significantly facilitate transmission of pathogens (Wikel, S. K. 1999. Tick modulation of host immunity: an important factor in pathogen transmission. Int J Parasitol 29:851-859Wikel, S. K. 1999. Tick modulation of host immunity: an important factor in pathogen transmission. Int J Parasitol 29:851-859). The result was the description of four tick salivary antigens whose vaccines partially protected from Ixodes ricinus or I. scapularis infestation or/and the pathogens they transmit (Prevot, P. P., B. Couvreur, V. Denis, M. Brossard, L. Vanhamme, and E. Godfroid. 2007. Protective immunity against Ixodes ricinus induced by a salivary serpin. Vaccine 25:3284-3292; Labuda, M., A. R. Trimnell, M. Lickova, M. Kazimirova, G. M. Davies, O. Lissina, R. S. Hails, and P. A. Nuttall. 2006. An antivector vaccine protects against a lethal vector-borne pathogen. PLoS Pathog 2:e27; de la Fuente, J., C. Almazan, U. Blas-Machado, V. Naranjo, A. J. Mangold, E. F. Blouin, C. Gortazar, and K. M. Kocan. 2006. The tick protective antigen, 4D8, is a conserved protein involved in modulation of tick blood ingestion and reproduction. Vaccine 24:4082-4095; Almazan, C., K. M. Kocan, E. F. Blouin, and J. de la Fuente. 2005. Vaccination with recombinant tick antigens for the control of Ixodes scapularis adult infestations. Vaccine 23:5294-5298; Decrem, Y., M. Mariller, K. Lahaye, V. Blasioli, J. Beaufays, K. Zouaoui Boudjeltia, M. Vanhaeverbeek, M. Cerutti, M. Brossard, L. Vanhamme, and E. Godfroid. 2008. The impact of gene knock-down and vaccination against salivary metalloproteases on blood feeding and egg laying by Ixodes ricinus. Int J Parasitol 38:549-560). The results presented herein demonstrate how the blood feeding ability of I. scapularis nymphs can be impaired by vaccinating the vertebrate blood donor against one of its arthropod salivary immunomodulators.

In certain cases, the protection achieved by the vaccination was not 100%, but this is not surprising given its dependence on the anti-sialostatin L2 titer of the animals. This dependence shows that there may be an underlying potential for further protection by increasing the anti-sialostatin L2 titer in the vertebrate hosts (e.g., by trying different vaccination protocols or the use of adjuvants). The results provided herein show that the observed effects on tick feeding may be attributed to blockage of sialostatin L2 activity.

Use of an anti tick vaccine against tick borne pathogens could be advantageous in that it would target the vector and therefore confer protection against all or substantially all the pathogens that this vector may transmit. It is additionally an environmentally friendly and cost effective method. Inclusion of sialostatin L2 in such a vaccine may be advantageous in an integrated approach to control disease transmission by I. scapularis bites in endemic areas. Apart from its effect on the tick population, there are some additional advantages revealed in the experiments described herein:

i) The observed boost of sialostatin L2 titer upon tick exposure and when compared with pre infestation titer suggests that there may be natural reminding doses of sialostatin L2 upon nymphal bites. Sialostatin L2 is expressed in adult ticks, too, upon their infestation of rabbits (Kotsyfakis, M., S. Karim, J. F. Andersen, T. N. Mather, and J. M. Ribeiro. 2007. Selective cysteine protease inhibition contributes to blood-feeding success of the tick Ixodes scapularis. J Biol Chem 282:29256-29263).

ii) Nymphs are efficient disease vectors, because their small size makes it difficult to notice their attachment. Indeed, a primary problem for physicians in endemic areas is that patients can not ‘feel’ an attached tick and they do not realize they have been bitten by ticks. The observed redness and edema formation in the vaccine group would make possible for timely detection of the attached tick and its removal.

iii) The observed increased inflammation and host immune reaction at tick attachment sites can make the vector host interface a considerably hostile environment for pathogens the vector transmits.

iv) The observed increased recognition of tick salivary gland antigens in the vaccine group can add sensitivity in detection of anti tick Abs.

Taken together, the experiments described herein demonstrate that Sialostatin L2, an important salivary player for tick feeding success escapes from the ‘attention’ of vertebrate immunity. Further, the work described herein uncovers another approach for the discovery of similarly ‘silent’ tick salivary antigens that can confer protection from tick bites. Accordingly, this idea may be used to develop vaccines and immunogenic compositions against other parasitic diseases as well.

### Methods

The above results were obtained using the following methods and materials.

Unless otherwise indicated, protocols followed standard procedures (11), and all experiments were performed at room temperature (25±1 C). All materials were obtained from Sigma (St. Louis, Mo.), and the water used was of 18 MΩ quality, produced by a MILLIQ apparatus (Millipore, Bedford, Mass.).

Bioinformatics tools. To obtain genomic information relative to the cystatin transcripts, raw trace fasta files from shotgun genomic sequences of I. scapularis I. scapularis (found in ftp://ftp.ncbi.nih.gov/pub/TraceDB/ixodes scapularis) representing early 24 million sequences were downloaded and removed of vector and primer sequences using a home-made tool written in Visual Basic. Sequences with average quality values below 20 were excluded. Sialostatins L and L2 coding sequences (NCBI accession gi:22164282 and gi:67083499, respectively) were blasted against these genomic sequences using blastn with a word size of 80 (−W 80 switch). The resulting matches were assembled using the cap3 assembler 12), and the produced consensus sequences were in turn blasted against the two cystatin transcripts. All other sequence comparisons reported here were done using the BLAST server at the NCBI (on the world wide web at ncbi.nlm.nih.gov/BLAST/) and the ClustalW Service at the European Bioinformatics Institute (on the world wide web at 2.ebi.ac.uk/-clustalw), while protein secretion signals were revealed in the SignalP 3.0 server (on the world wide web at cbs.dtu.dk/services/SignalP/) of the Technical University of Denmark.

Expression, purification, and sequence verification of sialostatin L2. The same procedure was followed as described before for sialostatin L (9) except that sialostatin L2 cDNA was PCR amplified using high-fidelity Taq polymerase from a λTriplEx2 cDNA clone, described previously (8), with gene-specific primers for subcloning into the pET17b bacterial expression vector as follows:

(Forward 5/-GCC CAT ATG GAA CTG GCA
CTG CGT GGC GGT TAC CGC GAG CG-3/
Re-verse 5/-GCC CTC GAG TTA TGC GGC
CGC ACA CTC AAA GGA GCT-3/) designed

Sialostatin L2 Preparation and LPS Decontamination. Sialostatin L2 gene was overexpressed in bacteria and the corresponding active protein purified in 0.8 mM stock solution (10 g/l) as previously described (9, 77). This stock solution was subjected to removal of any potential LPS contamination by Arvys Proteins Inc. (Stamford, Conn.) using a detergent based method. Samples were subjected to decontamination treatment five times, and endotoxin presence by the end of the procedure was estimated as lower than 0.00004 endotoxin units/μg of protein (roughly, less than 3×10 ⁻¹⁴ g of endotoxin/μg of protein) with a sensitive fluorescence based endotoxin assay (PyroGene recombinant Factor C endotoxin detection system; Lonza Biologics Inc., Portsmouth, N.H.). The protein concentration was then adjusted with sterile PBS in 1 g/l for vaccination of the animals.

Enzymatic assays Apparent inhibition constants of sialostatin L2 or L for various proteases were obtained as described earlier (9) by measuring loss of enzymatic activity at increasing concentrations of inhibitor in the presence of a fluorogenic enzyme substrate in large excess.

Production of polyclonal sera. Female Guinea pigs and female Swiss Webster mice, 6-8 weeks old, were purchased from the Jackson Laboratory (Bar Harbor, Me.) and maintained in the NIAID Animal Care Facility (Twinbrook 3 Building) under pathogen-free conditions in temperature-controlled rooms and receiving water and food ad libitum. Groups of six mice each received intradermal injections of 10 μg of pure recombinant protein in each ear, and four boosts followed at 2-wk intervals. Pre-immune sera were taken from each mouse prior to vaccination, while control groups received buffer (vehicle) vaccination in parallel. Guinea Pigs (GPs) were vaccinated intradermally with 100 micrograms of sialostatin L2 for 4 times in 2 weeks intervals. All treatments were performed in accordance with The Guide for Care and Use of Laboratory Animals (NIH).

Tick Exposure and Animal Handling. Pathogen free nymphal I. scapularis ticks were obtained from colonies maintained at Oklahoma State University. Ticks were maintained at 24° C. and 90% relative humidity under a 14:10 h photoperiod. Approximately 3 wks old (250 300 g) outbred female albino Hartley guinea pigs were obtained from Charles River Laboratories (Wilmington, Mass.). Guinea pigs were maintained and subjected to intradermal vaccination four times in two wks intervals in the left and right front lateral edge of their back with 50 μg of pure recombinant sialostatin L2 (see below) on each side (100 μg total) according to the approved guidelines of the National Institutes of Health Animal Care and Use Committee.

Two wks after the last vaccination and prior to tick placement, guinea pigs were sedated and the area between the ears was shaved. Twenty nymphal ticks were placed on the shaved area and allowed to attach. The head was completely covered with stockinet during sedation until all ticks were attached, verified by gentle pull using forceps. Ticks that did not attach by the time the guinea pig recovered from anesthesia were removed and subtracted from the total number of ticks placed. During tick attachment, guinea pigs were checked daily, and detached ticks were weighed and recorded. The animal cages were set in pans with water to ensure ticks could not escape, and nutrients provided ad libitum.

Tick rearing. For most experiments, ticks were harvested after detaching from mice (nymphs) or rabbits (adults). Engorged nymphs were maintained at 23° C. and >90% relative humidity under 14-h light/10-hdark photoperiod until enough time elapsed for them to molt into the adult stage. In all feeding experiments involving adult ticks, we placed an equal number of female and male ticks on the ears of New Zealand white rabbits. Ears were covered with cotton ear bags, and an Elizabethan collar was placed around the neck of each rabbit to prevent grooming. Engorged adult ticks were held under similar conditions as nymphs until enough time elapsed for them to lay eggs. For harvesting tick tissues, partially fed females were dissected within 4 h of being removed from hosts.

Harvesting tick tissues. Tick tissues (salivary glands and midguts) were dissected in ice-cold 100 mM 3-(N-morpholino)-propanesulfonic acid (MOPS) buffer containing 20 mM ethylene glycol bis-(β-aminoethyl ether)-N,N,N/,N/-tetraacetic acid (EGTA), pH 6.8. After removal, glands were washed gently in the same ice-cold buffer. Dissected tissues were stored immediately after dissection in RNALATER (Ambion, Austin, Tex.) prior to isolating total RNA. Tissues were used immediately after dissection or stored at −70° C. in 0.5 M piperazine N,N-bis-2-ethane sulfonic acid, pH 6.8, containing 20 mM EGTA, 1× Complete™ Mini Protease inhibitor cocktail (Roche, Indianapolis, Ind.). All other manipulations were carried out at 4° C.

Synthesis of tick salivary gland cDNA and reverse transcription-polymerase chain reaction (RT-PCR). Total RNA was isolated using an RNAqueous™ total RNA isolation kit (Ambion) from dissected partially fed female salivary glands/midguts and unfed female adult salivary glands/midguts. Concentration of total RNA was determined spectrophotometrically, aliquoted, and stored at −70° C. before use. Total RNA was reverse transcribed using Moloney murine leukemia virus reverse transcriptase according to manufacturer's protocol. For each gene, cDNA was PCR amplified using gene-specific primers: for sialostatin L2 Forward 5/-CTA TGC GGC TTC CTC GAA GGG GCT-3/ and Reverse 5/-GGC TAC AGC GAG AGG GCG AAC CAC CAA-3/; tick salivary gland Isac Forward 5/-AGC GAA GAC GGT CTC GAG CAA GAT-3/ and Reverse 5/-TCG GCA CAC GAT GCC TCA GGG AAT-3/; and β-actin Forward 5/-GAA GAT CTT GAG AAG ATG GCC CAG-3/ and Reverse 5/-CGG TAC CGT CGA TGG TCA CC-3/ as the control. The PCR program used included the following cycles: 75° C. for 3 min; 94° C. for 2 min; 22 cycles of 94° C. for 1 min, 49° C. for 1 min and 72° C. for 1.20 min; followed with 10 minutes at 72° C. Real-time quantitative RT-PCR (RT-qPCR)-RT-qPCR was performed using the Mx4000 or Mx3005P Multiplex Quantitative PCR System and the Brilliant SYBR Green Single-Step QRTPCR Master Mix Kit (Stratagene, La Jolla, Calif.) according to the manufacturer's instructions. A standard curve (100 to 107 copies per reaction) was generated using purified sialostatin L and L2 PCR products as the template. The following primers were used for all reactions: For sialostatin L Forward 5/-TCG CGA TCG CTA GCA TCA CAC TT-3/ and Reverse 5/-AGC AGA AGG ACC AAA GCG AAG GTA-3/; for sialostatin L2 Forward 5/-AAG TCC ATT AGC TCC TTC GAG TGT G-3/ and Reverse 5/-ATC ATT CCG CGA CGT ACA GTG AGA-3/. Reactions (25 μl final volume) contained 10 ng of total RNA and were run under the following conditions: 1 cycle of 50° C. for 30 min and 95° C. for 15 min, followed by 40 cycles of 95° C. for 30 sec and 55° C. 30 sec. Fluorescence was measured every cycle at the end of the 55° C. step. Samples were run in triplicate as well as in the absence of reverse transcriptase or template as negative controls. The copy number of sialostatin L and L2 mRNA in each sample was determined using the Mx4000 or Mx3005P data analysis software based on the standard curve.

Double-stranded (ds)RNA synthesis, tick injections, and feeding. Sialostatin L2 RT-PCR product was joined to the Block-iT T7 TOPO linker. This TOPO linking reaction was used in two PCR reactions with gene-specific and T7 PCR primers to produce sense and antisense linear DNA templates. These sense and antisense DNA templates were used to generate sense and antisense transcripts using the BLOCK-iT RNA TOPO transcription kit. The resulting dsRNA was analyzed by agarose gel electrophoresis to verify its size. Subsequently, unfed female ticks were injected with 0.5 μg cystatin dsRNA or with 1 μL TS.MOPS (vehicle) using a 35-gauge needle. After injection of dsRNA or buffer alone, ticks were kept at 37° C. overnight under high humidity to observe tick survival. Surviving ticks were exposed to a naïve (never tick-bitten) rabbit and allowed to blood feed to repletion. Their feeding success was determined by total engorged weight, survival, and egg lying. The ears of the rabbits exposed to dsRNA sialostatin L2 or water-injected ticks were cleaned by the end of the experiment; the animals were kept for 14 days and then re-exposed to normal unfed ticks, and feeding success evaluation was performed as described herein.

ELISA and Enzymatic Assays. ELISA titer was determined under standard methods (114) as the dilution of the primary Ab that gives an absorbance read at 405 nm twice higher than that of the negative control (pre immune serum from the same animal). As a secondary Ab, we used an alkaline phospatase conjugated goat anti guinea pig IgG diluted 5000 times in 50 mM Tris pH 8, 150 mM NaCl, 0.1% Tween 20 (TTBS), and the 96 well plate was incubated with p nitrophenyl phosphate liquid alkaline phosphatase substrate for 1 h at 37° C. before absorbance reading in a THERMOmax colorimetric plate reader (Molecular Devices, Sunnyvale, Calif.).

Enzymatic assays for cathepsin L inhibition were performed as previously described (9). Total IgGs were purified from guinea pig sera using the Melon™ gel IgG purification kit (Pierce, Rockford, Ill.) according to manufacturer's instructions and subsequently tested for neutralizing sialostatin L2 activity.

Statistics. Data are shown as mean±SEM where applicable. Statistical differences were analyzed by analysis of variance (ANOVA), KS test (Kolmogorov Smimov comparison of two data sets), χ2 test, and Student t test depending on their applicability to the hypothesis tested. A P value of 0.05 or less was considered statistically significant

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### LITERATURE CITED

1. Sauer, J. R., McSwain, J. L., Bowman, A. S., and Essenberg, R. C. (1995). Annu Rev Entomol 40, 245-267.
2. Ribeiro, J. M., and Francischetti, I. M. (2003). Annu Rev Entomol 48, 73-88.
3. Wikel, S. K., and Bergman, D. (1997). Parasitol Today 13, 383-389.
4. Wikel, S. K. (1999). Int J Parasitol 29, 851-859.
5. Anderson, J. F. (2002). Med Clin North Am 86, 205-218.
6. Estrada-Pena, A., and Jongejan, F. (1999). Exp Appl Acarol 32, 685-715.
7. Valenzuela, J. G., Francischetti, I. M., Pham, V. M., Garfield, M. K., Mather, T. N., and Ribeiro, J. M. (2002). J Exp Biol 205, 2843-2864.
8. Ribeiro, J. M., Alarcon-Chaidez, F., Francischetti, I. M., Mans, B. J., Mather, T. N., Valenzuela, J. G., and Wikel, S. K. (2006). Insect Biochem Mol Biol 36, 111-129.
9. Kotsyfakis, M., Sa-Nunes, A., Francischetti, I. M., Mather, T. N., Andersen, J. F., and Ribeiro, J. M. (2006). J Biol Chem 281, 26298-26307.
10. Karim, S., Miller, N. J., Valenzuela, J., Sauer, J. R., and Mather, T. N. (2005). Biochem Biophys Res Commun 334, 1336-1342.
11. Sambrook J., Fritish E. F., and Maniatis T (1989). Molecular Cloning, A Laboratory Manual (NY: Cold Spring Harbor Press).
12. Huang X, Madan A (1999). Genome Res 9, 868-877.
13. Williams, J. W., and Morrison, J. F. (1979). Methods Enzymol 63, 437-467.
14. Pedra, J. H., Narasimhan, S., Deponte, K., Marcantonio, N., Kantor, F. S., and Fikrig, E. (2006). Am J Trop Med Hyg 75, 677-682.
15. Schorderet, S., and Brossard, M. (1993). Med Vet Entomol 7, 186-192.
16. Vray, B., Hartmann, S., and Hoebeke, J. (2002). Cell Mol Life Sci 59, 1503-1512.
17. Bode, W., Engh, R., Musil, D., Thiele, U., Huber, R., Karshikov, A., Brzin, J., Kos, J., and Turk, V. (1988). EMBO J 7, 2593-2599.
18. Turk, V., and Bode, W. (1991). FEBS Lett 285,213-219.
19. Bjork, I., Brieditis, I., Raub-Segall, E., Pol, E., Hakansson, K., and Abrahamson, M. (1996). Biochemistry 35,10720-10726.
20. Grunclova, L., Horn, M., Vancova, M., Sojka, D., Franta, Z., Mares, M., and Kopacek, P. (2006). Biol Chem 387,1635-1644.
21. Zhou, J., Ueda, M., Umemiya, R., Battsetseg, B., Boldbaatar, D., Xuan, X., and Fujisaki, K. (2006). Insect Biochem Mol Biol 36,527-535.
22. Ribeiro, J. M. (1995). Infect Agents Dis 4,143-152.
23. Reddy, V. Y., Zhang, Q. Y., and Weiss, S. J. (1995). Proc Natl Acad Sci USA 92, 3849-3853.
24. Serveau-Avesque, C., Martino, M. F., Herve-Grepinet, V., Hazouard, E., Gauthier, F., Diot, E., and Lalmanach, G. (2006). Biol Cell 98,15-22.
25. Hsing, L. C.; and Rudensky, A. Y. (2005). Immunol Rev 207,229-241.
26. Zavasnik-Bergant, T., and Turk, B. (2006). Tissue Antigens 67,349-355.
27. Narasimhan, S., Montgomery, R. R., DePonte, K., Tschudi, C., Marcantonio, N., Anderson, J. F., Sauer, J. R., Cappello, M., Kantor, F. S., and Fikrig, E. (2004). Proc Natl Acad Sci USA 101,1141-1146.
28. Liu, J., Sukhova, G. K., Sun, J. S., Xu, W. H., Libby, P., and Shi, G. P. (2004). Arterioscler Thromb Vasc Biol 24,1359-1366.
29. Gocheva, V., and Joyce, J. A. (2007). Cell Cycle 6,60-64.
30. Ribeiro, J. M. (1995) Infect. Agents Dis. 4,143-152
31. Sauer, J. R., McSwain, J. L., Bowman, A. S., and Essenberg, R. C. (1995) Annu. Rev. Entomol. 40,245-267
32. Ribeiro, J. M., and Francischetti, I. M. (2003) Annu. Rev. Entomol. 48, 73-88
33. Wikel, S. K., and Bergman, D. (1997) Parasitol. Today 13,383-389
34. Gem, L., Schaible, U. E., and Simon, M. M. (1993) J. Infect. Dis. 167, 971-975
35. Valenzuela, J. G., Francischetti, I. M., Pham, V. M., Garfield, M. K., Mather, T. N., and Ribeiro, J. M. (2002) J. Exp. Biol. 205,2843-2864
36. Turk, B., Turk, D., and Turk, V. (2000) Biochim. Biophys. Acta 1477, 98-111
37. Honey, K., and Rudensky, A. Y. (2003) Nat. Rev. Immunol. 3,472-482
38. Lombardi, G., Burzyn, D., Mundinano, J., Berguer, P., Bekinschtein, P., Costa, H., Castillo, L. F., Goldman, A., Meiss, R., Piazzon, I., and Nepomnaschy, I. (2005) J. Immunol. 174,7022-7032
39. Reinheckel, T., Hagemann, S., Dollwet-Mack, S., Martinez, E., Lohmuller, T., Zlatkovic, G., Tobin, D. J., Maas-Szabowski, N., and Peters, C. (2005) J. Cell Sci. 118,3387-3395
40. Felbor, U., Dreier, L., Bryant, R. A., Ploegh, H. L., Olsen, B. R., and Mothes, W. (2000) EMBO J. 19,1187-1194
12. Reddy, V. Y., Zhang, Q. Y., and Weiss, S. J. (1995) Proc. Natl. Acad. Sci. U.S.A. 92,3849-3853
41. Serveau-Avesque, C., Ferrer-Di Martino, M., Herve-Grepinet, V., Hazouard, E., Gauthier, F., Diot, E., and Lalmanach, G. (2005) Biol. Cell 98,15-22
42. Wille, A., Gerber, A., Heimburg, A., Reisenauer, A., Peters, C., Saftig, P., Reinheckel, T., Welte, T., and Buhling, F. (2004) Biol. Chem. 385,665-670
43. Joyce, J. A., Baruch, A., Chehade, K., Meyer-Morse, N., Giraudo, E., Tsai, F. Y., Greenbaum, D. C., Hager, J. H., Bogyo, M., and Hanahan, D. (2004) Cancer Cell 5,443-453
44. Nomura, T., and Katunuma, N. (2005) J. Med. Invest. 52,1-9
17. Fossum, K., and Whitaker, J. R. (1968) Arch. Biochem. Biophys. 125, 367-375
45. Vray, B., Hartmann, S., and Hoebeke, J. (2002) Cell Mol. Life Sci. 59, 1503-1512
46. Hartmann, S., and Lucius, R. (2003) Int. J. Parasitol. 33,1291-1302
47. Karim, S., Miller, N. J., Valenzuela, J., Sauer, J. R., and Mather, T. N. (2005) Biochem. Biophys. Res. Conn⁻nun. 334,1336-1342
48. Sambrook, J., Fritish, E. F., and Maniatis T. (1989) Molecular Cloning: A Laboratory Manual, Cold Spring Harbor Press, Cold Spring Harbor, N.Y.
49. Altschul, S. F., Gish, W., Miller, W., Myers, E. W., and Lipman, D. J. (1990) J. Mol. Biol. 215,403-410
50. Thompson, J. D., Higgins, D. G., and Gibson, T. J. (1994) Nucleic Acids Res. 22,4673-4680
51. Page, R. D. (1996) Comput. Appl. Biosci. 12,357-358
52. Bendtsen, J. D., Nielsen, H., von Heijne, G., and Brunak, S. (2004) J. Mol. Biol. 340,783-795
53. Andersen, J. F., Champagne, D. E., Weichsel, A., Ribeiro, J. M., Balfour, C. A., Dress, V., and Montfort, W. R. (1997) Biochemistry 36,4423-4428
54. Bjork, I., Pol, E., Raub-Segall, E., Abrahamson, M., Rowan, A. D., and Mort, J. S. (1994) Biochem. J. 299,219-225
55. Pol, E., Olsson, S. L., Estrada, S., Prasthofer, T. W., and Bjork, I. (1995) Biochem. J. 311,275-282
56. Barrett A. J., Rawlings, N. D., and Woessner, J. F., Jr. (1998) Handbook of Proteolytic Enzymes, Vol. 2, pp. 1051-1416, Academic Press, London
56. Oliveira, F., Kamhawi, S., Seitz, A. E., Pham, V. M., Guigal, P. M., Fischer, L., Ward, J., and Valenzuela, J. G. (2005) Vaccine 24,374-390
57. Valenzuela, J. G., Charlab, R., Mather, T. N., and Ribeiro, J. M. (2000) J. Biol. Chem. 275,18717-18723
58. Otto, H. H., and Schirmeister, T. (1997) Chem. Rev. 97,133-172
59. Kirschke, H., Kembhavi, A. A., Bohley, P., and Barrett, A. J. (1982) Biochem. J. 201,367-372
60. Maciewicz, R. A., Etherington, D. J., Kos, J., and Turk, V. (1987) Collagen Relat. Res. 7,295-304
61. Schonemeyer, A., Lucius, R., Sonnenburg, B., Brattig, N., Sabat, R., Schilling, K., Bradley, J., and Hartmann, S. (2001) J. Immunol. 167,3207-3215
62. Dahl, S. W., Halkier, T., Lauritzen, C., Dolenc, I., Pedersen, J., Turk, V., and Turk, B. (2001) Biochemistry 40,1671-1678
63. Pham, C. T., and Ley, T. J. (1999) Proc. Natl. Acad. Sci. U.S.A. 96, 8627-8632
64. Wolters, P. J., Pham, C. T., Muilenburg, D. J., Ley, T. J., and Caughey, G. H. (2001) J. Biol. Chem. 276,18551-18556
65. Adkison, A. M., Raptis, S. Z., Kelley, D. G., and Pham, C. T. (2002) J. Clin. Invest. 109,363-371
66. Rowan, A. D., Mason, P., Mach, L., and Mort, J. S. (1992) J. Biol. Chem. 267, 15993-15999
67. Keyszer (1995) Arthritis Rheum. 38:976-984
68. Troen et al. (1991) Cell Growth Differ. 2:23-31
69. Nakagawa et al. (1998) Science 280:450-453
70. Mukherjee S, Ukil A, Das P K., Antimicrob Agents Chemother. 2007 May; 51(5):1700-7. Epub 2007 Mar. 5
71. Maurer M S, Burkhoff D, Fried L P, Gottdiener J, King D L, Kitzman D W J Am Coll Cardiol. 2007 Mar. 6; 49(9):972-81. Epub 2007 Feb. 20
72. Di Piazza M, Mader C, Geletneky K, Herrero Y Calle M, Weber E, Schlehofer J, Deleu L, Rommelaere J. J Virol. 2007 April; 81(8):4186-98. Epub 2007 Feb. 7
73. Chuo L J, Sheu W H, Pai M C, Kuo Y M Dement Geriatr Cogn Disord. 2007;23(4):251-7. Epub 2007 Feb. 19.
74. Ribiero J. M. C., Kotsyfakis M, Karim S., Andersen J., Mather T. N. JBC 2007. Submitted.
75. Trager, W. 1939. Acquired immunity to ticks. J. Parasitol., 25:57-81.
76. Sa-Nunes, A., A. Bafica, D. A. Lucas, T. P. Conrads, T. D. Veenstra, J. F. Andersen, T. N. Mather, J. M. Ribeiro, and I. M. Francischetti. 2007. Prostaglandin E2 is a major inhibitor of dendritic cell maturation and function in Ixodes scapularis saliva. J Immunol 179:1497-1505.
77. Kotsyfakis, M., S. Karim, J. F. Andersen, T. N. Mather, and J. M. Ribeiro. 2007. Selective cysteine protease inhibition contributes to blood-feeding success of the tick Ixodes scapularis. J Biol Chem 282:29256-29263.
78. Prevot, P. P., B. Couvreur, V. Denis, M. Brossard, L. Vanhamme, and E. Godfroid. 2007. Protective immunity against Ixodes ricinus induced by a salivary serpin. Vaccine 25:3284-3292.
79. Labuda, M., A. R. Trimnell, M. Lickova, M. Kazimirova, G. M. Davies, O. Lissina, R. S. Hails, and P. A. Nuttall. 2006. An antivector vaccine protects against a lethal vector-borne pathogen. PLoS Pathog 2:e27.
80. de la Fuente, J., C. Almazan, U. Blas-Machado, V. Naranjo, A. J. Mangold, E. F. Blouin, C. Gortazar, and K. M. Kocan. 2006. The tick protective antigen, 4D8, is a conserved protein involved in modulation of tick blood ingestion and reproduction. Vaccine 24:4082-4095.
81. Almazan, C., K. M. Kocan, E. F. Blouin, and J. de la Fuente. 2005. Vaccination with recombinant tick antigens for the control of Ixodes scapularis adult infestations. Vaccine 23:5294-5298.
82. Francischetti, I. M., J. G. Valenzuela, J. F. Andersen, T. N. Mather, and J. M. Ribeiro. 2002. Ixolaris, a novel recombinant tissue factor pathway inhibitor (TFPI) from the salivary gland of the tick, Ixodes scapularis: identification of factor X and factor Xa as scaffolds for the inhibition of factor VIIa/tissue factor complex. Blood 99:3602-3612.
83. Monteiro, R. Q., A. R. Rezaie, J. M. Ribeiro, and I. M. Francischetti. 2005. Ixolaris: a factor Xa heparin-binding exosite inhibitor. Biochem J 387:871-877.
84. Francischetti, I. M., T. N. Mather, and J. M. Ribeiro. 2003. Cloning of a salivary gland metalloprotease and characterization of gelatinase and fibrin(ogen)lytic activities in the saliva of the Lyme disease tick vector Ixodes scapularis. Biochem Biophys Res Commun 305:869-875.
85. Garg, R., I. J. Juncadella, N. Ramamoorthi, Ashish, S. K. Ananthanarayanan, V. Thomas, M. Rincon, J. K. Krueger, E. Fikrig, C. M. Yengo, and J. Anguita. 2006. Cutting edge: CD4 is the receptor for the tick saliva immunosuppressor, Salp15. J Immunol 177:6579-6583.
86. amamoorthi, N., S. Narasimhan, U. Pal, F. Bao, X. F. Yang, D. Fish, J. Anguita, M. V. Norgard, F. S. Kantor, J. F. Anderson, R. A. Koski, and E. Fikrig. 2005. The Lyme disease agent exploits a tick protein to infect the mammalian host. Nature 436:573-577.
87. Francischetti, I. M., T. N. Mather, and J. M. Ribeiro. 2005. Tick saliva is a potent inhibitor of endothelial cell proliferation and angiogenesis. Thromb Haemost 94:167-174.

1-71. (canceled)

72. A method for the detecting a tick infestation in a subject comprising:
administering to the subject an immunogenic composition comprising one or more cystatin antigens, wherein the cystatin corresponds to a polypeptide comprising an amino acid sequence which is at least 80% homologous to the amino acid sequence of SEQ. ID. NO. 2 and the subject was previously exposed to the immunogenic composition; and wherein the immunogenic composition is administered in an amount that stimulates a desired immune response to the immunogenic composition in the subject; thereby detecting tick infestation in a subject.

73. The method of claim 72, further comprising the step of monitoring the subject for an inflammatory response or a pain response, wherein an inflammatory response or a pain response indicates a tick infestation.

74. The method of claim 73, wherein an inflammatory response is indicated by red skin or swollen skin.

75. The method of claim 72 where ticks do not remain attached to the subject as a result of detecting the tick infestation.

76. The method of claim 72, where tick feeding is reduced or impaired as a result of detecting the tick infestation.

77. The method of claim 72, where transmission of tick borne pathogens is reduced or impaired as a result of detecting the tick infestation.

78. The method of any one of claims 72-74, wherein detecting tick infestation occurs less than 72 hours after tick infestation.

79. The method of any one of claims 72-74, wherein a pain response at a site of tick infestation is detected by the subject.

80. A method of decreasing the ability of a tick to feed on a subject comprising:
administering to the subject an immunogenic composition comprising one or more cystatin antigens, wherein the cystatin corresponds to a polypeptide comprising an amino acid sequence which is at least 80% homologous to the amino acid sequence of SEQ. ID. NO. 2; thereby decreasing the ability of a tick to feed on a subject.

81. A method for preventing a tick infestation in a subject comprising:
administering to the subject an immunogenic composition comprising one or more cystatin antigens, wherein the cystatin corresponds to a polypeptide comprising an amino acid sequence which is at least 80% homologous to the amino acid sequence of SEQ. ID, NO. 2; thereby preventing a tick infestation in a subject.

82. A method for decreasing a risk for a tick borne disease in a subject comprising:
administering to the subject an immunogenic composition comprising one or more cystatin antigens, wherein the cystatin corresponds to a polypeptide comprising an amino acid sequence which is at least 80% homologous to the amino acid sequence of SEQ. ID. NO. 2; and
monitoring the subject for an inflammatory response or a pain response wherein an inflammatory response or a pain response indicates a risk for tick-borne disease in a subject.

83. The method of claim 82, wherein the disease is a member selected from the group consisting of: Lyme Disease, Anaplasmosis, East Coast Fever, Babesiosis, or tick-borne Encephalitis.

84. The method of claim 83, further comprising the step of treating the subject with a therapeutic or prophylactic agent for Lyme Disease, Anaplasmosis, East Coast Fever, Babesiosis, or tick-borne Encephalitis.

85. A method for the prevention of a tick-borne disease in a subject comprising:
administering to the subject an immunogenic composition comprising one or more cystatin antigens, wherein the cystatin corresponds to a polypeptide comprising an amino acid sequence which is at least 80% homologous to the amino acid sequence of SEQ. ID. NO. 2; thereby preventing the tick-borne disease in a subject.

86. The method of claim 85, wherein the disease is a member selected from the group consisting of Lyme Disease, Anaplasmosis, East Coast Fever, Babesiosis, or tick-borne Encephalitis.

87. An immunogenic composition comprising one or more cystatin antigens in combination with one or more additional antigens derived from tick saliva, wherein the cystatin corresponds to a polypeptide comprising an amino acid sequence which is at least 80% homologous to the amino acid sequence of SEQ. ID, NO. 2.

88. The composition of claim 87 wherein the cystatin antigen is a member selected from the group consisting of proteins, recombinant proteins or DNA-based immunogenic compositions.
